(19) United States
(12) Patent Application Publication
(10) Pub. No.: US 2011/0027817 A1
(43) Pub. Date: Feb. 3, 2011

(54) TAU PROTEIN SCREENING ASSAY

(76) Inventors: Ottavio Arancio, New York, NY (US); Daniela Puzzo, Catania (IT); James G. Moe, Stamford, CT (US)

Correspondence Address: WilmerHale/Columbia University, 399 PARK AVENUE, NEW YORK, NY, 10022, US

(21) Appl. No.: 12/719,703

(22) Filed: Mar. 8, 2010

Related U.S. Application Data
(63) Continuation-in-part of application No. PCT/US2008/075584, filed on Sep. 8, 2008.
(60) Provisional application No. 60/967,924, filed on Sep. 7, 2007.

(51) Int. Cl. C12Q 1/02; G09B 19/00

(52) U.S. Cl. 435/29; 434/236

(57) ABSTRACT

This invention is directed to methods for determining if a test compound can ameliorate tau protein induced reduction of long term potentiation in a neural structure. The invention is also directed to methods for determining if a test compound can re-establish or rescue synaptic function in a neural structure following damage by tau proteins. Also encompassed by disclosures in this invention are methods to determine if a test compound can increase synaptic function in a neural structure contacted with tau proteins and methods for determining if a test compound is capable of treating Alzheimer's disease or other tauopathies in a subject.

This application is a continuation-in-part of International Application No. PCT/US2008/75584, filed Sep. 8, 2008, and claims the benefit of and priority to U.S. provisional patent application Ser. No. 60/967,924 filed Sep. 7, 2007, are each herein incorporated by reference in their entirety.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

The classical hallmarks of Alzheimer's disease (AD) are inter-neuronal plaques consisting of precipitates or aggregates of amyloid beta protein (Aβ), and intra-neuronal neurofibrillary tangles (NFTs) of tau protein. During AD progression, levels of extracellular tau increase indicated by levels of tau in the cerebrospinal fluid (CSF) (Formichi et al. 2006).

Plaques and tangles in AD and insoluble protein aggregates in other neurodegenerative diseases have been the primary focus of drug discovery efforts. Traditional target identification using animal and cell models, genomics and proteomics, cannot identify useful targets or drugs for inhibition of protein misfolding and aggregation, as the molecular events that cause protein misfolding and aggregation are distinct from the functions of traditional drug targets. Oligomer formation is therefore invisible to traditional pharmaceutical company target identification methods and there is a need for screening methods to evaluate the ability of test compounds to ameliorate conditions associated with protein oligomerization. This invention addresses these needs.

### SUMMARY OF THE INVENTION

In one aspect the invention relates to a method for determining whether a test compound can ameliorate tau protein induced reduction of long term potentiation in a neural structure, the method comprising: (a) contacting a neural structure with a solution containing tau proteins and a test compound, (b) stimulating long term potentiation between neurons in the a neural structure, (c) measuring long term potentiation between neurons in the neural structure, and (d) comparing the long term potentiation measured in (c) with long term potentiation measured in neurons in a neural structure contacted with tau proteins in the absence of a compound, wherein an increase in long term potentiation measured in (c) relative to long term potentiation measured in the absence of the compound indicates that the test compound ameliorates tau protein induced reduction of long term potentiation in a neural structure.

In another aspect, the invention relates to a method for determining whether a test compound can re-establish or rescue synaptic function in a neural structure following damage by tau proteins, the method comprising: (a) contacting a neural structure with a solution containing tau proteins and a test compound, (b) stimulating long term potentiation between neurons in the neural structure, (c) measuring long term potentiation between neurons in the neural structure, and (d) comparing the long term potentiation measured in (c) with long term potentiation measured in neurons in a neural structure contacted with tau proteins in the absence of a compound, wherein an increase in long term potentiation measured in (c) relative to long term potentiation measured in the absence of the compound indicates that the test compound re-establishes or rescues synaptic function in a neural structure following damage by tau proteins.

In yet another aspect, the invention relates to a method for determining whether a test compound can increase synaptic function in a neural structure contacted with tau proteins, the method comprising: (a) contacting a neural structure with a solution containing tau proteins and a test compound, (b) stimulating long term potentiation between neurons in the neural structure, (c) measuring long term potentiation between neurons in the neural structure, and (d) comparing the long term potentiation measured in (c) with long term potentiation measured in neurons in a neural structure contacted with tau proteins in the absence of a compound, wherein an increase in long term potentiation measured in (c) relative to long term potentiation measured in the absence of the compound indicates that the test compound increases synaptic function in a neural structure contacted with tau protein.

In still a further aspect, the invention relates to a method for determining whether a test compound is capable of treating Alzheimer's disease in a subject, the method comprising: (a) contacting a neural structure with a solution containing tau proteins and a test compound, (b) stimulating long term potentiation between neurons in the neural structure, (c) measuring long term potentiation between neurons in the neural structure, and (d) comparing the long term potentiation measured in (c) with long term potentiation measured in neurons in a neural structure contacted with tau proteins in the absence of a compound, wherein an increase in long term potentiation measured in (c) relative to long term potentiation measured in the absence of the compound indicates that the test compound is capable of treating Alzheimer's disease in a subject.

In yet another aspect, the invention relates to a method for determining whether a test compound can treat a tauopathy disorder in a subject, the method comprising: (a) contacting a neural structure with a solution containing tau proteins and a test compound, (b) stimulating long term potentiation between neurons in the a neural structure, (c) measuring long term potentiation between neurons in the a neural structure, and (d) comparing the long term potentiation measured in (c) with long term potentiation measured in neurons in a neural structure contacted with tau proteins in the absence of a compound, wherein an increase in long term potentiation measured in (c) relative to long term potentiation measured in the absence of the compound indicates that the test compound is capable of treating a tauopathy disorder in a subject.

In one embodiment of any of the aspects of the invention described herein, the neural structure is a hippocampal slice.

In another embodiment of any of the aspects of the invention described herein, the neural structure is contacted with the tau protein and test compound serially.

In a further embodiment of any of the aspects of the invention described herein, the neural structure is contacted with the tau protein and the test compound in parallel.

In still a further embodiment of any of the aspects of the invention described herein, neural structure is contacted with the tau protein and the test compound for about 30 minutes.

In another embodiment of any of the aspects of the invention described herein, the contacting step comprises perfusion of the neural structure.

In yet another embodiment of any of the aspects of the invention described herein, the method comprises an additional step before step (a) of acquiring a baseline long term potentiation reading.

In yet another aspect, the invention relates to a method for determining whether a test compound can treat a learning behavior defect in a subject, the method comprising: (a) administering a solution containing tau proteins to a first non-human mammal, (b) administering a test compound to the first non-human mammal, (c) measuring contextual learning behavior in the first non-human mammal, and (d) comparing the contextual learning behavior measured in (c) with contextual learning behavior measured in a second non-human mammal, wherein an increase in contextual learning behavior measured in (c) relative to the contextual learning behavior measured second non-human mammal indicates that the test compound is capable of a learning behavior defect in the subject.

In one embodiment of any of the aspects of the invention described herein, the second non-human mammal is administered with the solution containing tau proteins but not administered the test compound. In another embodiment of any of the aspects of the invention described herein, the tau proteins are administered into the hippocampus of the non-human mammal In another embodiment of any of the aspects of the invention described herein, the administration is through a cannula implanted into the hippocampus of the non-human mammal In another embodiment of any of the aspects of the invention described herein, the contextual learning is freezing behavior or fear conditioning.

In another embodiment of any of the aspects of the invention described herein, the learning behavior defect is Alzheimer's disease.

In still a further embodiment of any of the aspects of the invention described herein, the concentration of the tau protein is about 1 pM to about 5 μM.

In another embodiment of any of the aspects of the invention described herein, the tau proteins are monomers, soluble oligomers, insoluble aggregates or any combination thereof.

In another embodiment of any of the aspects of the invention described herein, the tau proteins are any of the six known isoforms and any derivatives including enzymatic and nonenzymatically modified tau molecules.

In another embodiment of any of the aspects of the invention described herein, the tau protein is in a mixture with other proteins associated with neurodegenerative diseases such as beta amyloid and alpha-synuclein. In another embodiment of any of the aspects of the invention described herein, the solution further contains one or more proteins associated with neurodegenerative diseases.

In another embodiment of any of the aspects of the invention described herein, the tau proteins are oligomers, including soluble oligomers.

In still a further embodiment of any of the aspects of the invention described herein, the tau proteins are aggregated, including insoluble aggregates of tau.

In still another embodiment of any of the aspects of the invention described herein, the tau proteins are 3R or 4R isoforms or any fragments thereof.

In another embodiment of any of the aspects of the invention described herein, the tau proteins are hyperphosphorylated.

In yet another embodiment of any of the aspects of the invention described herein, the tau proteins comprise a P301L mutation.

In another embodiment of any of the aspects of the invention described herein, the tau proteins comprise a mutation that affects microtubule binding by the tau protein.

In another embodiment of any of the aspects of the invention described herein, the tau proteins are reactive monomers or reactive oligomers.

In another embodiment of any of the aspects of the invention described herein, the tau proteins are selected from the group consisting of tau 4R/2N, tau 4R/1N, or C-terminal tau 4R.

In another embodiment of any of the aspects of the invention described herein, the tau proteins have an intermolecular disulfide linkage.

In another embodiment of any of the aspects of the invention described herein, the tau proteins have an intramolecular disulfide linkage.

In another embodiment of any of the aspects of the invention described herein, the tau proteins are human Alzheimer's disease tau proteins.

In another embodiment of any of the aspects of the invention described herein, the tau proteins are phosphorylated at tyrosine 217, tyrosine 231, or any combination thereof.

In another embodiment of any of the aspects of the invention described herein, the tau proteins comprise a mutation that increases microtubule binding by the tau protein.

In another embodiment of any of the aspects of the invention described herein, the tau proteins comprise a mutation that decreases microtubule binding by the tau protein.

In still a further embodiment of any of the aspects of the invention described herein, the test compound is a small molecule, a molecule from a molecular compound library, a protein, a peptide, a nucleic acid, a synthetic molecule, a carbohydrate, a peptidomimetic, a lipid, an antibody or the like.

In still another embodiment of any of the aspects of the invention described herein, wherein more than one test compound is contacted to the neural structure.

In still another embodiment of any of the aspects of the invention described herein, the method comprises an additional step of contacting the neural structure with a kinase inhibitor.

In still a further embodiment of any of the aspects of the invention described herein, the method comprises an additional step of contacting the neural structure with a phosphatase inhibitor.

### BRIEF DESCRIPTION OF THE FIGURES

FIG. 1. Soluble oligomers are the acutely toxic structures of amyloid proteins. Histopathology of brain from AD patient using Thioflavin S staining for amyloid. FIG. 1A. Amyloid plaque consisting primarily of Aβ fibrils. FIG. 1B. Neurofibrillary tangles composed of tau filaments.

FIG. 2. Schematic representation of the biomolecules that play a role in the pathology of Alzheimer's disease and targets that are being exploited for drug discovery. Soluble oligomers of tau protein and Aβ have both been shown to be neurotoxic, whereas amyloid plaques consisting of deposited Aβ and neurofibrillary tangles consisting of aggregated tau protein may be a cellular response that sequesters these neurotoxic oligomer intermediates. Adapted from Roberson and Mucke, 100 Years and Counting: Prospects for Defeating Alzheimer's disease. Science 2006. 314: 781-784.

FIG. 3. Tau oligomer accumulation in the brain correlates with AD Progression. Post-mortem analyses of brain specimens from patients show increased levels of tau oligomers during AD progression. Biochemical methods were used to purify the granular tau oligomers from brain specimens. Atomic force microscopy was used for detection and quantification of the size and number of oligomers.

FIG. 4. Effect of soluble tau on CA1-LTP. Perfusion for 20 min with a solution containing tau protein markedly reduced potentiation at 120 minutes after induction of long term potentiation (LTP) through tetanic stimulation of the Schaeffer collateral pathway. The three arrows represent the 3 series of theta-burst tetanic stimulation. The bar represents the perfusion with tau oligomers.

FIG. 5. Silver-stained gel of tau 412 protein preparation. SDS-PAGE was performed on a 4-20% Tris-HCl (Biorad) at 80 volts for 30 min, followed by 60 min at 230 volts. Gel was washed twice in deionized water, and then fixed in 50% Methanol/10% Acetic acid for 10 mins. Gel was then stained with SilverXpress (Invitrogen). Lane 1, tau protein, predominantly monomer. Lane 2, tau protein oligomers post-incubation.

FIG. 6. ELISA for tau oligomers detects tau oligomers in cerebral spinal fluid (CSF) from AD patients. Detection of tau oligomers in CSF. CSF specimens from the New York Brain Bank (NYBB) at Columbia University Medical Center (CUMC) were analyzed with an ELISA (capture: mAbT46; reporter: A11) to measure tau oligomers. The results were grouped according to disease severity and support the biological relevance of extracellular tau oligomers in AD. Tau oligomer levels are indicated by relative fluorescence units (RFU) on the y-axis.

FIG. 7. Tau oligomer formation assay. FIG. 7A. Size exclusion chromatography was performed on tau protein incubated with polyanion or without polyanion. The control reaction only shows tau protein monomer and dimer. FIG. 7B. Time course of tau oligomer formation with polyanion showing rapid formation of tau oligomers In the absence of polyanion, no oligomers formed.

FIG. 8. Screening compounds inhibiting tau oligomer formation.

FIG. 9. Validation of tau oligomer formation using antibody A11 specific for oligomer conformation. TRQ=Tau412 incubated with RNA. TC=Tau 412 incubated in buffer.

FIG. 10. Tau oligomers accumulate in animal models in association with memory loss and are also found in extracts from patients with AD and frontotemporal dementia and parkinsonism linked to chromosome 17 (FTDP-17).

FIG. 11. Tau reduction prevented water maze deficits in mice over-expressing Aβ. Representative path tracings of mice in water maze 24 hours after completion of 3 days of hidden-platform training are shown. Mice with normal levels of tau and Aβ (Tau^{+/+}) remembered the position of the hidden platform better than mice with normal levels of tau but over-expressing Aβ (hAPP/Tau^{+/+}). However, mice over-expressing Aβ but lacking the tau gene (hAPP/Tau^{-/-}) performed even better than Tau^{+/+} mice.

FIG. 12. Gel filtration of tau412 monomer (lower trace) and oligomer showing an apparent molecular weight of 515 kD for the tau oligomer.

FIG. 13. Tau constructs and oligomer preparations. FIG. 13A. Schematic of tau constructs used and their nomenclature. E2 and E3 represent the alternatively spliced exons in the amino-terminal region of tau. R1-4 and R′ represent the four microtubule binding repeats and pseudorepeat, respectively. The numbering scheme is based on the longest isoform (4R/2N) and consistent with this numbering, the actual length of the 4R/1N isoform is 412 amino acids. FIG. 13B. Bar diagram of full length tau isoform tau 441 (4R/2N). FIG. 13C. Schematic of non-reactive monomer, reactive monomer and reactive oligomer (a dimer is shown as an example). Reactivity implies the ability to form an intermolecular disulfide linkage. Intramolecular disulfide linkage formation in tau monomer precludes reactivity. The free thiols in a reactive monomer allow formation of an intermolecular or intramolecular disulfide linkage. Reactive oligomer has one or more free thiols available for monomer tau addition for oligomer extension by disulfide linkage. FIG. 13D. Non-reducing SDS-PAGE analysis of recombinant tau monomer and oligomer preparations. Lane 1. Oligomerized preparation of tau412, Lane 2. Unreactive Tau412 monomer purified from the oligomerized preparation shown in lane 2. FIG. 13E. Amino and carboxyl tau construct analysis by non-reducing SDS-PAGE. N-terminal tau was unable to oligomerize (Lane 1), whereas C-terminal tau readily formed oligomers (Lane 2).

FIG. 14. Analysis of the effect of oligomer enriched tau 4R on LTP. FIG. 14A. Perfusion with 50 nM oligomeric Tau 4R/2N dramatically reduced LTP (n=6 slices from 6 mice, F_{(1,14)}=21.86; p=<0001 compared to 10 vehicle treated slices from 10 mice). Each bar denotes the mean±SEM in this and the following figures. FIG. 14B. Concentration-response curve for the effect of tau 4R/2N on CA1-LTP (n=6 to 10 slices from 6 to 10 mice per each tau concentration). The shaded area corresponds to the average potentiation (continuous line) and the s.e. range in vehicle-treated slices. The residual potentiation was calculated by averaging the last 5 minutes of LTP in this and the following graphs. FIG. 14C. The graph demonstrates the average LTP data for different tau preparations, including oligomeric tau 4R/1N (100 nM and 2 μM), C-terminal tau 4R, N-terminal tau 1N, monomeric tau 4R/1N (n=6 to 10 slices from 6 to 10 mice for each tau construct; p<0.005 for oligomeric tau 4R/1N (100 nM and 2 μM) compared to vehicle-treated slices; p<0.001 for C-terminal tau 4R compared to vehicle-treated slices, and p>0.05 for N-terminal tau 1N compared to vehicle-treated slices).

FIG. 15. Tau oligomers impair associative fear memory. FIG. 15A. Non-reducing SDS-PAGE analysis of human tau isolated from severe AD brain tissue with tau pathology. Lanes 1 and 2 indicate tau dimer formation before and after 1 hr of incubation at 60° C., respectively. FIG. 15B. Immunoblot analysis of the human tau preparation. Non-reducing SDS-PAGE analysis of AD tau (Lane 1) indicates formation of tau dimer following incubation for 1 hr. at 60° C. (Lane 2). FIG. 15C. Purified AD tau is phosphorylated. Immunoblot analysis of recombinant tau441 (Lane 1) and Tau purified from AD brain (Lane 2) shows specific interaction of tau phospho-epitope-specific antibodies against tyrosines at amino acid positions 231 and 217 with AD tau but no reactivity with recombinant tau, whereas phospho-independent monoclonal antibody HT7 against total tau interacts with both recombinant and AD tau, as expected. FIG. 15D. Bilateral injections of oligomeric tau 4R/2N (500 nM), or C-terminal tau 4R (500 nM), or oligomeric tau 4R/1N (500 nM), in a final volume of 1 μl over 1 min—but not N-terminal tau (500 nM) or monomeric tau 4R/1N (500 nM)—into dorsal hippocampi, 180 and 20 min prior to the training, impaired contextual conditioning performance as the mice were exposed to the context after 24 hrs. Freezing responses during the training phase (baseline) were not affected by the treatment with different tau preparations. Vehicle-infused control mice: n=18, oligomeric tau 4R/2N: n=11, C-Terminal tau 4R: n=11, N-terminal Tau 1N: n=10, monomeric tau 4R/1N: n=11, oligomeric tau 4R/1N: n=4 (baseline: p>0.05; 24h contextual: p<0.01 for tau 4R/2N and C-terminal tau 4R compared to control, p<0.01 for tau 4R/1N compared to control). FIG. 15D. Bilateral injections of oligomeric human AD tau (500 nM) in a final volume of 1 μl over 1 min into dorsal hippocampi, 180 and 20 min prior to the training, impaired contextual fear memory. Freezing responses during the training phase were not affected by the treatments. Vehicle-infused control mice: n=13, oligomeric human tau (preparation 1): n=11, oligomeric human tau (preparation 2): n=13 (baseline: p>0.05; 24 h contextual: p<0.01 in mice infused with two different preparations containing oligomeric human AD tau compared to control vehicle-infused mice).

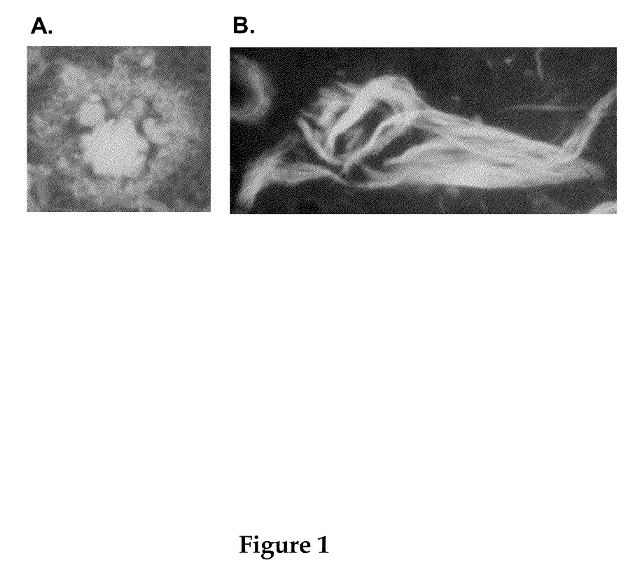

### DETAILED DESCRIPTION OF THE INVENTION

The issued patents, applications, and other publications that are cited herein are hereby incorporated by reference to the same extent as if each was specifically and individually indicated to be incorporated by reference.

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The invention provides methods of screening to identify compounds in an ex vivo assay using hippocampal slices exposed to soluble tau oligomers. The in vivo screening methods of the invention can be used for the evaluation of behavioral deficits, histopathologic and biochemical changes due to overexpression of mutated tau in P301L mice. In one aspect, the invention provides methods to screen compounds in a tau oligomer in-vitro assay from a library of compounds. In one embodiment the library can comprise compounds that inhibit tau fibrillation in-vitro. In another aspect, the invention provides methods to test the ability of compounds to inhibit or dissociate tau oligomers. In another aspect, the invention provides methods test ability of compounds that successfully inhibit or dissociate oligomers and have good toxicity profiles to rescue synaptic function in an ex vivo assay using hippocampal slices exposed to soluble tau oligomers to select compounds for in vivo testing. In another aspect aspect, the invention provides for screening assays for identifying a compound that reduces the neurotoxic effects of tau oligomers in hippocampal slices. In another aspect, the invention provides for an in vivo screening assay to counteract behavioral changes due to tau oligomers. In another aspect, the invention provides an assay for screening test compounds that ameliorate the impact of soluble tau. The assay can be used to evaluate test compounds is a dose-dependent manner.

In another aspect, the invention provides a tool to discover the mechanism by which tau inhibits LTP, and potentially new targets (for example, receptors that mediate the toxicity of extra-cellular tau) for drug discovery for AD and other tauopathies.

### Tau Protein

Tau protein is a 50-64 kDa neuronal microtubule-associated protein (MAP), which is expressed predominantly in the axons of the central (CNS) and peripheral (PNS) nervous system neurons. Tau proteins play an important role in the assembly of tubulin monomers into microtubules and function to constitute neuronal microtubule networks, and establish links between microtubules and other cytoskeletal elements. By inducing bundling and stabilization of cellular microtubules, tau promotes neurite outgrowth in addition to its role in establishing and maintaining neuronal cell polarity. Tau expression is developmentally regulated by an alternative splicing mechanism and six different isoforms exist in the human adult brain. The CNS isoforms are generated by alternative mRNA splicing of 11 exons. Alternative splicing of Exons 2 (E2), 3 (E3) and 10 (E10) gives rise to six tau isoforms that range from 352 to 441 amino acids. These isoforms include tau352, tau 383, tau381, tau410, tau412 and tau 441. The isoforms differ in whether they contain three (tau-3L, tau-3S or tau-3: collectively 3R) or four (tau-4L, tau-4S or tau-4: collectively 4R) tubulin-binding domains/repeats (R) of 31 or 32 amino acids each at the C-terminal They also differ on whether they have two (tau-3L, tau-4L), one (tau-3S, tau-4S) or no (tau-3, tau-4) repeats of 29 amino acids each in the N-terminal portion of the molecule. The terminal repeat sequences are encoded by exons 9, 10, 11 or 12. As tau is developmentally regulated, only the shortest tau isoform (tau-3) is expressed in the fetal brain, but all six isoforms are seen in the adult human brain. Tau's interactions with microtubules are mediated by the tubulin-binding domains/repeat at the C-terminal region.

In one aspect, the biological activity of tau is regulated by phosphorylation. Tau phosphorylation is developmentally regulated and fetal tau is more highly phosphorylated in the embryonic compared to the adult CNS and the degree of phosphorylation of the six adult tau isoforms decreases with age. Although the relative importance of individual sites for regulating the binding of tau to microtubules is unclear, phosphorylation at some sites, such as Serine-262 and Serine-396, has been reported to play a dominant role in reducing the binding of tau to microtubules. Both sites are phosphorylated in fetal tau and they are hyperphosphorylated in all six adult human brain tau isoforms that form paired helical filaments (PHFs) in Alzheimer's disease (AD). Hyperphosphorylation dislodges tau from the microtubule surface, resulting in compromised axonal integrity and accumulation of toxic tau peptides.

The major candidate tau kinases include mitogen-activated protein kinase, glycogen synthase kinase 3b, cyclin-dependent kinase 2 (cdk2), cyclin-dependent kinase 5, cAMP-dependent protein kinase, Ca²⁺ sub/calmodulin-dependent protein kinase II, microtubule-affinity regulating kinase and stress-activated protein kinases. Although glycogen synthase kinase-3 may function as the predominant tau kinase in the brain, there are a number of other kinases that have been implicated in the phosphorylation of tau. Moreover, protein phosphatases counterbalance the effects of tau kinases. For example, inhibition of protein phosphatases by okadaic acid in cultured human neurons results in increased tau phosphorylation, decreased tau binding to microtubules, selective destruction of stable microtubules and rapid axonal degeneration. In addition to phosphorylation, tau is also subject to regulation by ubiquitination, nitration, truncation, prolyl isomerization, association with heparan sulfate proteoglycan, glycosylation, glycation and modification by advanced glycation end-products.

### Mutants of Tau

Different mutations impair protein function, promote tau fibrilization or perturb tau gene splicing, leading to aberrant and distinct tau aggregates. To date, two types of mutations have been identified in autosomal dominant tauopathies—intronic mutations that disrupt the splicing of tau and missense mutations that alter the function of tau. The relative proportion of the 3R-tau and 4R-tau isoforms is regulated by splicing and there is evidence that loss of this normal regulation can contribute to tau aggregation. Moreover, transgenic tau mutant mice exhibit behavioral and neuropathological defects and tau aggregation is sufficient to cause a dementia in mice and in humans.

Missense, deletion or silent mutation in the coding region or intronic mutations in the tau gene occur in cases of frontotemporal dementia with Parkinsonism (FTDP-17) and indicate a link to the regulation of cognitive neurology. Coding region mutations located in, or close to, the microtubule binding repeat region reduce the ability of tau to interact with microtubules whereas, intronic mutation can result in increased 4R isoform expression levels leading to filamentous tau pathologies. Three separate families with frontotemporal dementia, having the same molecular mutation in Exon 10 of tau gene (P301 L) have been described (Bird et al,). Nevertheless, differences in clinical features and pathologic outcomes are observed among individuals carrying the same mutation indicating that environmental and genetic factors can also influence pathologies related to tau dysfunction.

### Taupathies

As described herein, although there is a significant link to cognitive neurology, tau pathology is not restricted to the central nervous system alone. Clusters (“tangles”) of paired-helical filaments containing phosphorylated tau is observed in inclusion body myositis. Tau pathology has also been described in myotonic dystrophy. Adult patients with myotonic dystrophy Type 1 (DM1) frequently develop a focal dementia with aging, agreeing with recent studies documenting an abnormal tau-protein expression in the brain tissues of patients with DM1.

Although its identification as major component of the neurofibrillary tangle implicated tau in the pathogenesis of AD, tau protein dysfunction is now known to contribute to a number of diseases which are functionally grouped as “taupathies” (Table 1). There is a significant overlap of clinical features between taupathies. Neurofibrillary tangles (NFT) occur in AD, FTDP-17, progressive supranuclear palsy (PSP), whereas neuropil threads occur in AD, Cortico basalganglionic degeneration (CBD), FTDP-17 and PSP. Techniques such as silver impregnation techniques and immunohistochemistry with monoclonal antibodies against phosphorylated or nonphosphorylated epitopes of tau can be used to detect most of the tau inclusions. Tau-positive glial inclusions are also observed in both oligodendrocytes and astrocytes. The four classes of tau aggregation have been described include 1) AD and Parkinsonism dementia complex (six tau isoforms); 2) PSP and CBD (the three isoforms with Exon 10 corresponding sequence); 3) Pick's disease (PiD) (the three isoforms without Exon 10) and 4) myotonic dystrophy- the shortest tau isoform.

TABLE 1
Tauopathies

Dementia Syndromes
Alzheimer's Disease
Tangle Only Dementia
Argyrophyllic grain disease
Pick's disease
Presenile dementia with tangles and calcification
Progressive subcortical gliosis
FTDP-17
Subacute sclerosing panencepalitis
Gertermann-straussler-scheinker disease with tangles
Prion protein amyloid angiopathy
Parkinsonism plus syndromes
Corticobasal degeneration
Progressive supranuclear palsy
Multiple system atrophy
Postencephlatic Parkinsonism
Dementia pugilistica
Neuromuscular disorders
Amyotrophic lateral sclerosis with Parkinsonism
Dementia complex of Guam
Non-Guanamanian motor neuron disease with
neurofibrilary tangles
Myotonic dystrophy
Inclusion body myositis
Genetic/metabolic disorders
Down's syndrome
Hallervorden-spatz disease
Niemann-Pick disease type-c

### Tau in Parkinson's Disease and Parkinson's Syndromes

Tau protein deposition has been identified as a contributor to degenerative Parkinson's syndromes including progressive supranuclear palsy (PSP), Cortico basalganglionic degeneration (CBD), postencephalitic and posttraumatic Parkinsonism, FTDP-17 and Parkinsonism dementia complex of Guam as well in the original parkin mutation family. Tau deposition varies in topography, ultrastructure and protein chemistry in various diseases. Subcortical destruction in globus pallidus, subthalamic nucleus and midbrain/pontine reticular formation and homogenous depletion of substantia nigra pars reticulate in Progressive supranuclear palsy. Damage of globus pallidus as well as substantia nigra has been demonstrated in CBD, Parkinsonism dementia complex of Guam and postencephalitic Parkinsonism. The NFT in PSP is made of straight filaments and predominantly 4R tau. The isoform(s) of tau expressed in various diseases is shown in Table 2. The morphology of tangles varies with the isoform. When all six isoforms are expressed, they are paired helical filaments, while in 4R diseases they are twisted ribbon filaments (as in PiD) or straight filaments (as in PSP). Moreover, in many of these diseases, tau pathology has been described in glial cells as well.

TABLE 2
Tau neurodegenerative diseases and tau isoforms

| | All Isoforms | 4R Tau | 3R Tau |
|---|---|---|---|
| Familial | Alzheimer's disease, | Frontotemporal | Myotonic |
| | frontotemporal dementia | dementia | dystrophy |
| Sporadic | Alzheimer's disease, | Progressive | Frontotemporal |
| | Parkinsonism dementia | supranuclear | dementia (Pick's |
| | complex of Guam, | palsy, | disease) |
| | Postencephalitic | corticobasal | |
| | Parkinsonism | degeneration | |

### Tau in AD

Alzheimer's disease (AD) is characterized by the appearance of extracellular senile plaques and intracellular neurofibrillary tangles in the brain. Senile plaques are composed of the beta amyloid peptide whereas, neurofibrillary tangles contain paired helical filaments (PHF) that are formed of hyperphosphorylated isoforms of tau. Moreover, while the distribution of senile plaques is variable from one individual to another, neurofibrillary tangles are formed in a reproducible pattern. They first appear in the entorhinal cortex (EC) and spreads to surrounding areas such as the hippocampus. There is a correlation between the progress of the formation of tangles and the progress of the disease. Increases of tau phosphorylation are also observed during this progression.

The AS is further histopathologically characterized by reduced synaptic density and neuronal loss in selected brain areas. Tau pathology corresponds to the intraneuronal association of tau proteins into abnormal filaments and, more recently, with the extracellular aggregation of tau. In the AD brain, tau is abnormally hyperphosphorylated and is present mostly as PHF. Ultrastructurally neurofibrillary lesions contain pair helical lesions as a major fibrous component and straight filaments (SF) as a minor component. Both types are formed of the six brain tau isoforms in the hyperphosphorylated and abnormally phosphorylated form.

### Cerebrospinal Tau Extracellular

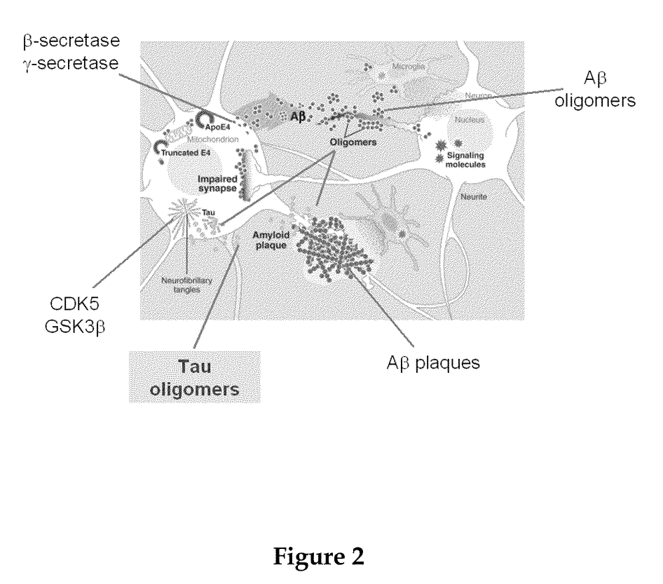

The concept that oligomers, small soluble aggregates of amyloid proteins (shown schematically in FIG. 2), are pathological entities in many amyloid diseases is an emerging paradigm finding increasing experimental support from recent published studies on proteins tau, Aβ, α-synuclein, and prion protein whose aggregation is associated with neurodegenerative diseases (Kayed et al. 2003, Selkoe 2005, Haas and Selkoe, 2007). Oligomers are in solution, and can mediate molecular interactions with other molecules (i.e. enzymes, receptors, pores, cell membrane etc), whereas Aβ plaques and tau tangles are more inert.

Current research indicates that the soluble fraction of tau protein is most acutely toxic in animal models of tauopathy (SantaCruz et al. 2005; Leroy et al. 2007; Yoshiyama et al. 2007) and that accumulation of multimeric species of tau correlates well with disease progression in the murine model conditionally expressing tau P301L (Berger et al. 2007). Results from studies of human CSF from AD and non-AD individuals support the observation that soluble, extracellular, oligomeric species of tau correlate with disease progression. Studies with murine hippocampal slices show an inhibitory effect of extracellular, soluble tau on synaptic plasticity and are consistent with a causative pathological role for extracellular, soluble tau species. Reduction of levels of tau protein has beneficial effects on behavior in murine models overproducing Aβ (Roberson et al 2007) or tau P301L (Asuni et al. 2007). Chemical compounds reducing levels of soluble tau oligomers, or compounds inhibiting the neurotoxic mechanism(s) of soluble tau oligomers may have efficacy for AD and other tauopathies. Compounds can be selected for their ability to 1) Inhibit the formation of tau oligomers or disrupt them, 2) Reverse inhibition of synaptic LTP caused by tau oligomers and 3) Ameliorate behavioral deficits in the tau P301L murine model.

An inverse relationship exists between the number of extracellular tangles and the number of surviving cells in damaged regions of the brain. One consequence is that neurons containing fibrillar lesions can degenerate and release the NFT into the extracellular environment. Similarly, upon neuron death, other intracellular components, like tau, can be released into the extracellular space and subsequently into the cerebrospinal fluid (CSF). Extracellular NFT can accumulate in the extracellular space and prove toxic to surrounding undamaged cells. Under physiological conditions, essentially all tau protein (>95%) is tightly bound to the microtubule (MT) network. However, in pathological conditions, tau could be modified by aberrant post-translational events, for example by hyperphosphorylation. These modifications can result in tau detaching from MTs and accumulating in a free form which then pass into the extracellular space when neurons degenerate. At high concentrations, tau self-aggregation may take place thereby further exacerbating the pathological condition. Moreover, tau, can exist in fragmented forms and three to five months may be required for elevated CSF tau levels to return to normal after an acute stroke. The rate of clearance of tau from the CSF in patients with neurodegenerative dementia, is also unknown

The role of CSF tau in the diagnosis of dementias has been examined using ELISA methods. Elevated CSF tau levels are associated with AD pathology and can be used as a candidate diagnostic biomarker. In one study, a 84%, positive predictive value of 87% and positive likelihood ratio of 5.3 in distinguishing patients with AD from cognitively normal controls was observed with a cutoff value of 234 pg/ml CSF tau. Phosphorylated tau levels in CSF have also been found to be useful as a biological marker of AD. Elevated CSF tau has also been reported in CBD, FTD and in many patients with Creutzfeldt-Jakob disease (CJD). It has been shown that higher amounts of phosphorylated tau in the CSF in sporadic CJD is associated with a rapid progression of the disease to akinetic mutism.

### Generation and Quantification of Tau Oligomers

A standardized simple procedure can be used to generate, identify and quantify tau-oligomers in vitro. The method is based on the ability of RNA facilitator to form oligomers of Prion protein (Vasan et al., 2006). The procedure involves incubating tau with RNA (RQ11+12) facilitates the formation of oligomers. The reaction occurs at physiological pH and temperature and can proceed to completion in 30-60 minutes. For example, U.S. patent application Ser. No. 11/704,079 describes methods for generating soluble tau oligomer formation and stabilization at low tau concentration at physiological pH and temperature with the use of polyanion molecules.

### Screening of Compounds for Disruption of Tau Oligomers

Efforts to systematically identify valid drug targets for neurodegenerative diseases, a first step toward finding effective drugs therapies, have failed to date because the underlying mechanisms of these diseases were not well understood. The major technical hurdle in the development of new drugs is the identification of disease-specific biological targets upon which drugs can act to ameliorate the disease process. The identification of therapeutic targets requires specific knowledge of a disease's etiology at the molecular and cellular level, and the appropriate animal model(s) for subsequent development of lead compounds which act upon the target.

Compounds binding to and altering the distribution of Tau oligomers can disrupt their toxicity. This can occur via several different mechanisms: 1) compounds can stabilize monomers and inhibit formation of oligomers that are neurotoxic; 2) compounds can bind to toxic oligomers and block their detrimental interaction at neurons (i.e. block tau oligomer receptor binding or tau oligomer pore formation); 3) in binding to toxic oligomers, compounds can promote their breakdown into smaller, non-toxic species; and 4) metabolism/clearance of tau protein can be facilitated by compounds which promote a shift towards monomeric tau. Anti-tau oligomer drug candidates that function through one or more of these pathways have substantial potential to be developed into disease modifying drugs (DMDs).

Recent work indicates that soluble oligomers, possibly trimers (three identical proteins irreversibly bound to one and other) (Townsend, 2006) or dodecamers (12 identical proteins irreversibly bound to one another) (Lesné, 2006) in the case of Aβ, are the primary mediators of neurotoxicity and accelerating Aβ fibrillization in mouse models for AD reduces functional deficits due to the reduction of soluble oligomers. Thus, drugs reducing Aβ fibrils at the cost of increasing soluble Aβ oligomers could be harmful (Cheng et al. 2007).

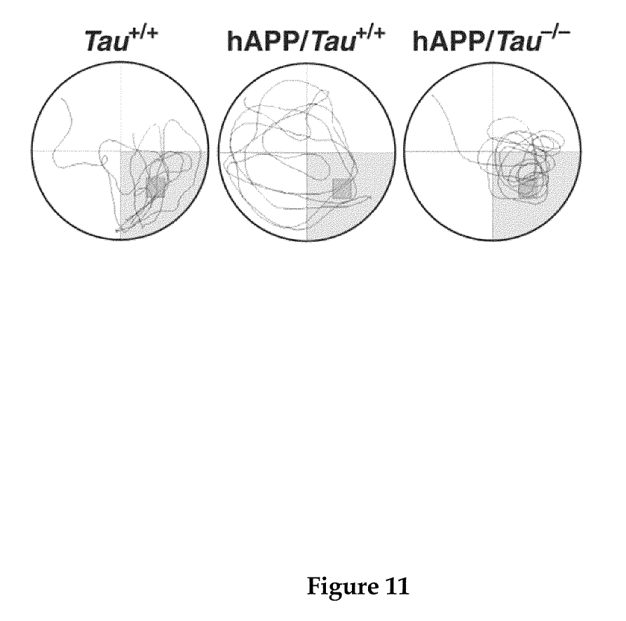

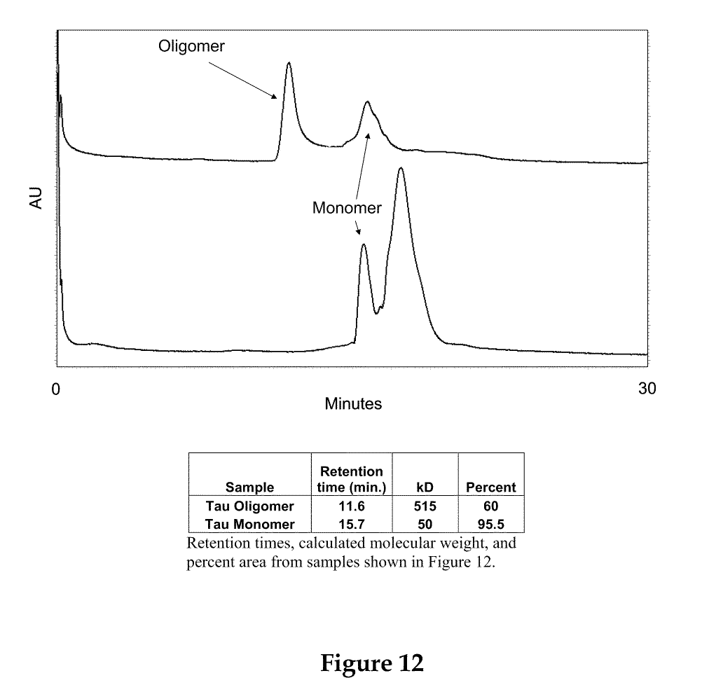

Validation of tau protein as a drug discovery target for AD is strongly supported by recent experiments in a mouse model for AD showing that reduction of tau levels protected mice from Aβ-induced cognitive impairments (FIG. 11, Roberson et al. 2007; see Boston Globe Article; See Science article, “A New Take on Tau”). Tau reduction also protected both transgenic and nontransgenic mice against excitotoxicity. The authors conclude that tau reduction can block Aβ- and excitotoxin-induced neuronal dysfunction and can represent an effective strategy for treating AD and related conditions. Therapeutic approaches for reduction of phosphorylated tau include pharmacologic manipulation of cellular protein refolding/degradation pathways (Goryunov et al. 2007) and immunotherapy (Asuni et al. 2007). (For a very recent review of tau-mediated neurodegeneration in AD and related disorders see Ballatore et al. 2007).

Binding to and altering the distribution tau and other amyloid aggregates can disrupt their toxic action on neurons. This can happen in a number of different ways, for example: 1) compounds can stabilize monomers or aggregates smaller than the one(s) that induce neurotoxicity, thereby reducing the pool of toxic aggregates; 2) compounds can bind to toxic aggregates and block their detrimental interaction at neurons; 3) in binding to toxic aggregates, compounds can promote their breakdown into smaller, non-toxic aggregates; 4) metabolism/clearance of Aβ can be facilitated by compounds which promote a shift to smaller aggregate sizes. Anti-Aβ drug candidates which function through one or more of these pathways include tramiprosate (Kisilvesky, Szarek & Weaver, 1997-2005; Gervais et al., 2006), and isomers of inositol (McLaurin et al., 2006).

### Test Compounds

Test compounds can be small molecules, molecules from a molecular compound library, proteins, peptides, nucleic acids, synthetic molecules, carbohydrates, peptidomimetics, lipids, or the like. Test compounds can also be antibodies. Several tau binding antibodies have been described and these include those antibodies described in table 3. However, because memory loss is a major clinical hallmark of AD and other tauopathies, compounds that not only interfere with tau-induced synaptic dysfunction but also improve tau-induced memory deficit can be assayed. Similarly, test compounds can also be assessed for their ability to inhibit tau oligomer formation and tau phosphorylation in tau P301L mice.

TABLE 3
Tau binding antibodies

| Antibody | Epitope Specificity/Application | Source |
|---|---|---|
| mAb HT7 | 159-163/Total Tau Determination | Pierce (MN1000) |
| mAb BT2 | 194-198/Total Tau Determination | Pierce (MN1010) |
| mAb T46 | Tau with C-terminus/Tau Oligomers | Invitrogen |
| mAB Tau C3 | C-Terminus of Tau Truncated at | Santa Cruz |
| | Asp 421 | Biotechnology |
| mAb AT8 | Phosphorylated Ser202 and Thr205 | Pierce |
| mAb PS422 | Phosphorylated Tau Ser422 | Biosource |
| | | International |
| mAb 6C5 | GAPDH Internal Control for Even | Biodesign |
| | Loading | |

Compounds that are assayed in methods described herein can be randomly selected or rationally selected or designed. The compound can be chosen randomly without considering the structure of other identified active compounds. An example of randomly selected compounds is the use a chemical library, a peptide combinatorial library, a growth broth of an organism, or a plant extract. The compound can also be chosen on a nonrandom basis. Rational selection can be based on the target of action or the structure of previously identified active compounds. Specifically, compounds can be rationally selected or rationally designed by utilizing the structure of compounds that are presently being investigate for use in treating Alzheimer's disease.

The test compounds can be, as examples, peptides, small molecules, and vitamin derivatives, as well as carbohydrates. The test compounds may be nucleic acids, natural or synthetic peptides or protein complexes, or fusion proteins. They may also be antibodies, organic or inorganic molecules or compositions, drugs and any combinations of any of said agents above. They may be used for testing, for diagnostic or for therapeutic purposes. A skilled artisan can readily recognize that there is no limit as to the structural nature of the test compounds to be used in accordance with the present invention.

Several compounds, including for example, general test molecules, such as curcumin, (−)-Epicatechin-3-gallate, Azure B, morin and/or analogs or derivatives thereof, can be tested with the methods of the invention.

Tau oligomers can be purified and compounds can be screened for disruption of oligomer structure. For example, tau oligomers can be isolated from Tau 412+RNA facilitator reaction mixture by size exclusion chromatography. Sephacryl S-300 column (1×31cm) can be calibrated using high molecular weight standards (Amersham). Tau oligomer reaction mixture can be chromatographed on the column and 1 ml fractions can be collected at a predefined time interval, for example every 6 minutes. Standard sandwich ELISA using antibodies against a tau epitope, for example mAb MN1000 (Pierce) against amino acids 159-163 or mAb MN1010 (Pierce) against tau epitope amino acids 194-198, can used to detect fractions containing tau peaks. The molecular weights of the oligomer peaks can be calculated from the standard curve. Fractions containing the oligomer peak can be pooled and concentrated. Compounds can be screened for converting oligomer structures to monomers using the ELISAs described herein.

The selection of compounds for future synthesis can be guided by structure-activity data gleaned from ongoing biological testing. Consideration can also be given to molecular diversity—by synthesizing and testing diverse structures to increase the likelihood of finding optimum anti-amyloidogenic activity in a chemical scaffold that affords a good PK profile. Syntheses can involve Stille or Suzuki coupling of a suitably protected bicyclic aromatic (e.g. 1-tosyl-3-stannylindole, 2-methoxynapthalene-6-boronic acid) to an arylbromide or aryliodide (e.g. 2-bromoquinoline, 6-iodonapthalen-2-ol). Alternatively, for 3,3′-linked bi-indole compounds, a substituted indole can be reacted with a substituted isatin. Reduction with borane then yields a 3,3′-bi-indole compound, which can then be further derivatized if desired.

Transmission electron microscopy (TEM) assays can be used to measure inhibition of Tau paired helical filament (PHF) formation. For example, frozen aliquots of Tau441 (8.3 mg/mL, 60 μL) in Tris buffer (50 mM, pH 7.4) can be thawed and diluted to 4 μM with Tris buffer (50 mM, pH 7.4). After incubating for 72 hrs, can be analyzed by TEM using the procedure of Cohen et al. (Biochemistry 2006, 45: 4727-35). Briefly, a 10 μL sample can be placed on a 400 mesh copper grid covered by carbon-stabilized Formvar film and allowed to stand for 60 s. Excess fluid can then be removed and the grids negatively stained for 60 s with uranyl acetate (10 μL, 2% solution). Excess fluid can again be removed, the grids dried for 30 min. and the samples viewed using an electron microscope operating at 80 kV. Active compounds reduce the number and/or alter the morphology of tau PHFs relative to controls.

Thioflavin S (ThS) Assays can be used to measure inhibition of Tau aggregation. For example, Tau441 can be prepared and stored at −78° C. as frozen aliquots (8.3 mg/mL, 60 μL) in Tris buffer (50 mM, pH 7.4) until used. To determine whether compounds inhibit tau441 aggregation, the protein (4 μM) can be incubated in buffer (50 mM Tris, pH 7.4, 1 mM dithiothreitol, 5 μM Thioflavin S (ThS)) at 37° C. for up to 48 h in a Tecan Genios microplate reader. To induce fibrillization, buffer can contain heparin (4 μg/mL). Incubations can be made in black 96-well polystyrene microplates by adding tau solution (200 μL) followed by compound stock solutions in DMSO (2 μL), or vehicle. Fluorescence can be measured every 15 min. (λₑₓ=450 nm, λₑₘ=480 nm) after shaking for 15 sec. and pausing 10 sec. before taking measurement. Compounds that inhibit tau aggregation can be identified by an attenuation in the increase in fluorescence relative to control incubations.

### Hippocampal Slices

In one aspect, the invention provides methods to assess the effect of soluble Tau monomers and oligomers (all soluble multimeric species of tau), and insoluble Tau aggregates on long term potentiation and synaptic dysfunction in Alzheimer's disease by perfusion of hippocampal slices with a solution containing tau.

As used herein, a brain slice refers to sections or explants of brain tissue which are maintained in culture. A skilled artisan can readily employ art known brain slice culture methods for use in the present invention. Brain slice culture can employ sections of whole brain tissue or explants obtained from specific regions of the brain. Any region can be used to generate an brain slice culture, including, but not limited to brain slice culture explants obtained from specific regions of the brain, for example the hippocampus region.

Any mammal can be used as a tissue source for the explant that is used to generate the brain slice culture used in the present method so long as the animal can serve as a tissue source and the slice culture can be established and maintained for a period sufficient to conduct the present methods. Such mammals include, but are not limited to humans, rats, mice, guinea pigs, monkeys and rabbits. The method of the invention may further comprise the step of obtaining a brain slice from the mammal, or providing a brain slice culture. The method may further comprise the step of culturing or cultivating the brain slice prior to transduction or transfection.

The mammal used as a tissue source can be a wild-type mammal or can be a mammal that has been altered genetically to contain and express an introduced gene. For example, the animal may be a transgenic animal, such as a transgenic mouse, that has been altered to express neural production of the β-amyloid precursor protein (Quon et al. (1991) Nature 35:598-607; Higgins et al. (1995) Proc Natl Acad Sci USA 92:4402-4406). In one embodiment, the mammal used as a tissue source is not a transgenic mammal that has been altered genetically to express tau protein or a variant thereof.

The mammal used as a tissue source can be of any age. In one embodiment, the mammalian tissue source will be a neonatal mammal.

Entire brain tissue can be used to establish an brain slice culture. Alternatively, a specific area or region of the brain can be used as an explant source. The preferred regions for the source of the brain slice culture are the hippocampus and cortex.

A variety of procedures can be employed to section or divide the brain tissues. For example, sectioning devices can be employed. The size/thickness of the tissue section will be based primarily on the tissue source and the method used for sectioning/division.

### Long Term Potentiation

LTP is an increase in the strength of a chemical synapse that lasts from minutes to several days. It is considered one of the major mechanisms by which memories are formed and stored in the brain. LTP has been observed both in experimental preparations in vitro and in living animals (in vivo). Under experimental conditions, applying a series of short, high-frequency electric stimuli to a synapse can strengthen, or potentiate, the synapse for minutes to hours. In living cells, LTP occurs naturally and can last from hours to days, months, and years. LTP has been observed in a variety of other neural structures, including the cerebral cortex, cerebellum, amygdala, and many others. LTP can even occur at all excitatory synapses in the mammalian brain (Malenka R, Bear M (2004) Neuron 44 (1): 5-21).

Neurons connected by a synapse that has undergone LTP have a tendency to be active simultaneously: after a synapse has undergone LTP, subsequent stimuli applied to one cell can elicit action potentials in the cell to which it is connected. LTP is believed to contribute to synaptic plasticity in living animals, providing the foundation for a highly adaptable nervous system. Because changes in synaptic strength can underlie memory formation, LTP is believed to play a critical role in behavioral learning.

The type of LTP exhibited between neurons depends in part upon the anatomic location in which LTP is observed because of the variety of signaling pathways that contribute to LTP. For example, LTP in the Schaffer collateral pathway, where axon collaterals given off by CA3 pyramidal cells in the hippocampus project to area CA1 of the hippocampus, is NMDA receptor-dependent, whereas LTP in the mossy fiber pathway is NMDA receptor-independent. As a result of its predictable organization and readily inducible LTP, the CA1 hippocampus has become the prototypical site of mammalian LTP study. NMDA receptor-dependent LTP in the adult CA1 hippocampus is the most widely studied type of LTP.

NMDA receptor-dependent LTP can be induced experimentally by applying a few trains of high-frequency stimuli (tetanic stimuli) to the connection between two neurons or to a pre-synaptic cell. In addition to strong tetanic stimulation of a single pathway to a synapse, LTP can also be induced cooperatively via the weaker stimulation of many. When one pathway into a synapse is stimulated weakly, it produces insufficient postsynaptic depolarization to induce LTP. In contrast, when weak stimuli are applied to many pathways that converge on a single patch of postsynaptic membrane, the individual postsynaptic depolarizations generated can collectively depolarize the postsynaptic cell enough to induce LTP cooperatively.

Chemical synapses are functional connections between neurons throughout the nervous system. In a typical synapse, information is passed from the first (presynaptic) neuron to the second (postsynaptic) neuron via a process of synaptic transmission. Through experimental manipulation, a non-tetanic stimulus can be applied to the presynaptic cell, causing it to release a neurotransmitter—typically glutamate—onto the postsynaptic cell membrane. There, glutamate binds to AMPA receptors (AMPARs) embedded in the postsynaptic membrane. The AMPA receptor is one of the main excitatory receptors in the brain, and is responsible for most of its rapid, moment-to-moment excitatory activity.[17] Glutamate binding to the AMPA receptor triggers the influx of predominantly sodium ions into the postsynaptic cell, causing a short-lived depolarization called the excitatory postsynaptic potential (EPSP).

The magnitude of this depolarization determines whether LTP can be induced in the postsynaptic cell. While a single stimulus does not generate an EPSP capable of inducing LTP, repeated stimuli given at high frequency cause the postsynaptic cell to be progressively depolarized as a result of EPSP summation: with each EPSP reaching the postsynaptic cell before the previous EPSP can decay, successive EPSPs add to the depolarization caused by the previous EPSPs. In synapses that exhibit NMDA receptor-dependent LTP, sufficient depolarization unblocks NMDA receptors (NMDARs), receptors that allow calcium to flow into the cell when bound by glutamate. While NMDARs are present at most postsynaptic membranes, at resting membrane potentials they are blocked by a magnesium ion that prevents the entry of calcium into the postsynaptic cell. Sufficient depolarization through the summation of EPSPs relieves the magnesium blockade of the NMDAR, allowing calcium influx. The rapid rise in intracellular calcium concentration triggers the maintenance and expression of LTP, specifically its early phase.

### Measuring LTP

Hippocampal slice preparations can be performed as described (Son et al, Learn Mem. 1998 July-August; 5(3):231-45), Briefly, mice (C57/B16) can be decapitated, and their hippocampi can be removed. Transverse hippocampal slices of a thickness of 400 μm can be made on a tissue chopper and transferred to an interface chamber, where they can be maintained at 29° C. They can be perfused (1-3 ml/min) with saline solution (124.0 mM NaCl/4.4 mM KCl/1.0 mM Na₂HPO₄/25.0 mM NaHCO3/2.0 CaCL2/2.0 mM MgSO₄/10 mM glucose) continuously bubbled with 95% O2 and 5% CO₂. Slices can be permitted to recover for at least 90 min before recording. A concentric bipolar platinum-iridium stimulation electrode and a low-resistance glass recording microelectrode filled with saline solution (5 mΩ resistance) can be placed in CA1 stratum radiatum to record the extracellular field excitatory postsynaptic potential (fEPSP).

An input-output curve can be used to set the baseline fEPSP at ≈35% of maximal slope. Baseline stimulation can be delivered every minute (0.01-ms duration pulses) for 15 min before beginning the experiment to assure stability of the response. Different concentrations of tau or tau oligomers can be added to perfused slices for 20 min in interleaved experiments. LTP can be induced by using θ-burst stimulation (4 pulses at 100 Hz, with the bursts repeated at 5 Hz and each tetanus including three 10-burst trains separated by 15 s). Responses can be recorded for 1 h after tetanization. Data analysis can be performed with a factorial ANOVA with post hoc correction. The results can be expressed as mean±SEM.

This system can be used as an ex vivo assay for screening compounds that ameliorate the impact of soluble tau on LTP that can represent a principle neurotoxic effect of extracellular tau protein. The advantages of this assay are that compound effectiveness can be evaluated prior to going into more testing in transgenic models of Alzheimer's disease and other tauopathies. Furthermore, targeting extracellular tau protein can represent an early step in the neuropathology of AD and other tauopathies giving it distinct advantages over current methodologies that target tau tangles.

Candidate compounds can be screened on the basis of their ability to re-establish normal LTP. The hippocampal slice assay can include a measure of LTP levels in the presence and absence of test compounds in slices treated with tau oligomers. Tau oligomers can be obtained from commercial sources or chemically synthesized. For example, following acquisition of a 15 minute baseline, slices can be perfused with a solution containing tau oligomers that have been pre-incubated for 30 minutes with the selected test compound. In separate experiments, slices can be treated with tau alone. Additional control slices can be treated with a solution that does not comprise tau proteins. Other slices can be treated with the test compound alone. If no difference is observed in levels of LTP between compound treated slices perfused with tau oligomers and vehicle-treated slices, it can be concluded that the compounds can re-establish or rescue synaptic function following damage by tau oligomers in hippocampal slices. Similarly, if there is an increase in synaptic function in slices treated with a test compound relative to those that that are not treated with a test compound, this can indicate the tests compound can increase synaptic function. These experiments can be performed with the use of synthetic tau, or with naturally produced tau. Test compounds can be screened on the basis of their ability to reduce LTP dampening effects of tau mutants, tau fragments of truncation products or tau phosphoproteins. Given that these experiments relate to the effect of test compounds on synthetic tau, the effect of these compounds on natural tau can be further characterized to determine if such compounds have a beneficial effect on synaptic dysfunction in tau P301L mice. Such analysis can be performed, for example, by determining whether basal synaptic transmission and LTP are impaired in the transgenic (Tg) mice. Based on the observation that tau P301L mice show NFT-related synaptic alterations (Katsuse et al 2006), a reduction in BST and/or LTP in slices from Tg mice may be observed with a 20 min perfusion with the test compound to determine if it rescues the synaptic deficit. Controls can be performed on slices from P301L mice treated with vehicle, and WT mice treated with compound or vehicle. If any test compounds re-establish normal synaptic function LTP in Tg slices, they can be selected for further behavioral testing.

Given that changes in synaptic strength can underlie changes in memory, infusion of tau oligomers onto the dorsal hippocampus, an area of the brain with remarkable plastic characteristics of the kind that are required for learning and memory, can impair memory behavioral performance in mice. In another aspect, the invention provides methods to assess the effect of soluble Tau, Tau aggregates and Tau oligomers on learning and memory by infusion of the dorsal hippocampus with a solution containing tau. Candidate compounds can be screened on the basis of their ability to re-establish normal memory. Mice can be anesthetized with 20 mg/kg Avertin and implanted with a 26-gauge guide cannula into the dorsal part of the hippocampi (coordinates: P=2.4 mm, L=1.5 mm to a depth of 1.3 mm) (Paxinos, G. (1998). Mouse brain in stereotaxic coordinates (New York, Academic Press). The cannulas can be fixed to the skull with acrylic dental cement (Paladur). The infusion can be made after 6-8 days through infusion cannulas that can be connected to a microsyringe by a polyethylene tube. The entire infusion procedure can take ˜1 min, and animals can be handled gently to minimize stress. Tau oligomers plus compound, Tau alone, or vehicle or compound alone can be injected bilaterally in a volume of 1 μl over 1 min. After injection, the needle can be left in place for another minute to allow diffusion. Tau oligomers can be obtained from commercial sources or chemically synthesized. For example, Tau oligomers pre-incubated for 30 minutes with the selected test compound or mixed with selected test compound at the time of the infusion, or tau oligomers alone, or vehicle or compound alone, can be infused 15 minutes prior to performing training for memory fear conditioning in a conditioning chamber. This associative memory test is much faster than other behavioral tasks that require multiple days of training and testing (Gong, B., O. V. Vitolo, F. Trinchese, S. Liu, M. Shelanski, and O. Arancio, Persistent improvement in synaptic and cognitive functions in an Alzheimer mouse model following rolipram treatment. J. Clin. Invest., 2004. 114: p. 1624-1634). The conditioning chamber can be placed be in a sound-attenuating box. The conditioning chamber can have a 36-bar insulated shock grid removable floor. For the cued and contextual conditioning experiments, mice can be placed in the conditioning chamber for 2 min before the onset of a discrete tone (CS) (a sound that lasted 30 s at 2800 Hz and 85 dB). In the last 2 s of the CS, mice can be given a foot shock (US) of 0.50 mA for 2 s through the bars of the floor. After the CS/US pairing, the mice can be left in the conditioning chamber for another 30 s and can then be placed back in their home cages. Freezing behavior, defined as the absence of all movement except for that necessitated by breathing, can be scored using the Freezeview software (MED Ass. Inc.). To evaluate contextual fear learning, freezing can be measured for 5 min (consecutive) in the chamber in which the mice can be trained 24 hr after training To evaluate cued fear learning, following contextual testing, the mice can be placed in a new context (triangular cage with smooth flat floor and with vanilla odorant) for 2 min (pre-CS test), after which they can be exposed to the CS for 3 min (CS test), and freezing can be measured. Sensory perception of the shock can be determined through threshold assessment, as described (Gong, B., O. V. Vitolo, F. Trinchese, S. Liu, M. Shelanski, and O. Arancio, Persistent improvement in synaptic and cognitive functions in an Alzheimer mouse model following rolipram treatment. J. Clin. Invest., 2004. 114: p. 1624-1634). If no difference is observed in percent of freezing between compound treated mouse infused with tau oligomers and vehicle-treated mice, it can be concluded that the compounds can re-establish or rescue memory function following damage by tau oligomers in mice. Similarly, if there is an increase in memory function in mice treated with a test compound relative to those that that are not treated with a test compound, this can indicate the tests compound can increase memory function.

The following examples illustrate the present invention, and are set forth to aid in the understanding of the invention, and should not be construed to limit in any way the scope of the invention as defined in the claims which follow thereafter.

### Examples

### Example 1

### Accumulation of Tau Oligomers and AD Progression

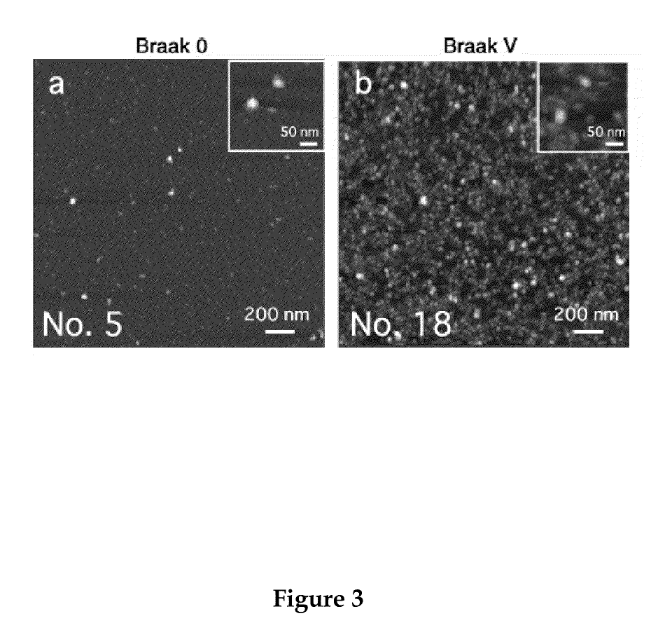

Recent publications show that over-expression of soluble tau caused neuron loss and memory impairment in mouse models for tauopathies, whereas NFTs were not associated with these phenotypes (SantaCruz et al. 2005; Spires et al. 2006; Yoshiyama et al. 2007). To determine if neurotoxic tau protein species can form before the development of tangles. Postmortem studies of tau oligomers in brain specimens during Braak stages 0-V. of AD were performed. The results showed a correlation between the accumulation of tau oligomers and AD progression (FIG. 3; Maeda et al., 2006).

### Example 2

### Tau Oligomers Cause Synaptic Dysfunction

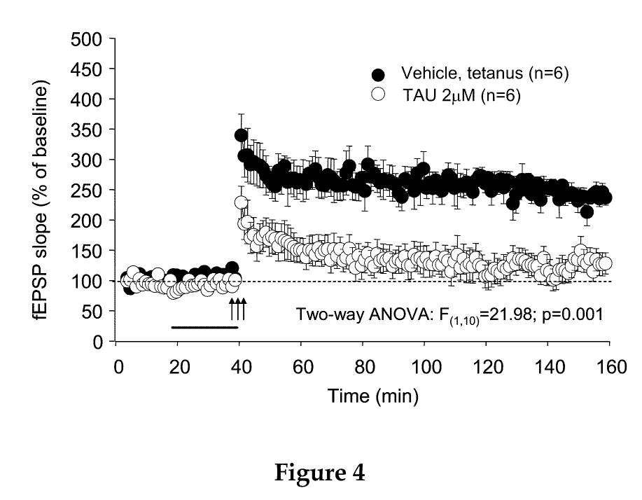

AD begins as a synaptic disorder that involves progressively larger areas of the brain over time (Masliah, 1995). To determine whether soluble extracellular tau in general or soluble tau oligomers specifically cause synaptic dysfunction a series of experiments were conducted. The experiments tested whether a brief application of tau oligomers was capable of producing a defect in LTP at the connection between Shaeffer collateral and CA1 pyramidal neurons in hippocampus of 3-5 month-old WT mice. Hippocampal slices were perfused with a solution containing tau protein (412 aa isoform) (2 μM) for 20 min before inducing LTP through tetanic stimulation of the Schaeffer collateral pathway. Potentiation in vehicle-treated slices was far greater than in tau-treated slices (levels of LTP: tau-treated slices equal to ˜130% at 120 min. after tetanus, vs ˜250% in vehicle-treated slices, n=6 for both, Two-way ANOVA: F(1,10)=21.98; p=0.001. (FIG. 4)

Dose response experiments can be performed and the percentage of tau oligomers in the ACSF perfusion buffer can be determined before it is applied t the hippocampal slices in order estimate the concentrations and specific tau structures inhibiting LTP. These experiments can be conducted with different tau isoforms and different portions of tau protein at a concentration of from about 1 pM to about 5 μM.

### Example 3

### Tau Protein Preparation

The cDNA for Tau412 was purchased from OriGene and subcloned into the bacterial expression vector pET21B to produce the tau412 protein with a C-terminal 6× His tag.

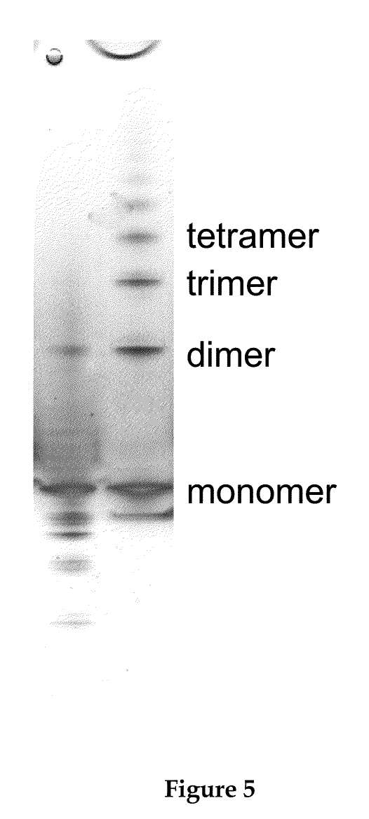

The bacterial strain BL21(DE3) was used for protein expression. Standard protocols from the cell and vector distributor (Novagen) were used to grow the cells and express the protein. Briefly, cells were streaked on LB Ampicillin plate and a single colony was picked and grown overnight in 2 ml 2XYT medium with glucose and 100 mg/ml carbenicillin. The overnight culture was used to inoculate a 500 ml culture which was grown to an optical density of 0.8 U/ml at 600 nm wavelength. Protein expression was induced with 1 mM IPTG for 4 hours at which time cells were pelleted at 4° C. by centrifugation at 6000 g. Pellets were stored overnight at −80° C. Cell lysates were prepared with Cell Lytic B lysis buffer lysozyme, benzonase and protease inhibitors according to manufacturer's protocol (Sigma). Cation exchange resin SP-Sepharose (GE Healthcare) was used for the first step of purification and 300 mM NaCl was used to elute tau protein. The second purification step used His-bind resin (Novagen) to bind the his-tagged tau protein which was eluted with imidazole. Buffer exchange into 50 mM tris-HCl pH 7.4 was performed with Amicon Ultra Centrifugal Devices (Millipore). Protein concentration was determined with BCA assay (Biorad). Tau oligomers were generated by incubation of 5 uM tau in Tris buffer pH 7.4 buffer with 100 mM NaCl at 37° C. FIG. 5 shows representative tau 412 protein preparation before and after incubation to form oligomers.

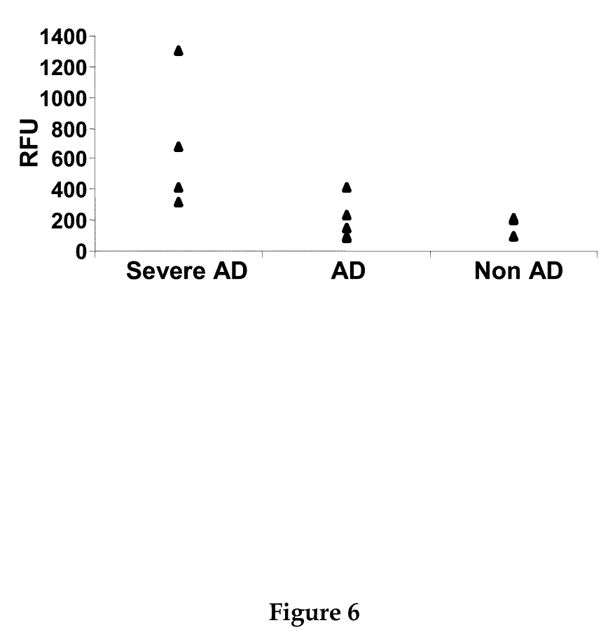

The formation of oligomers can be confirmed by immunoassays using various antibodies and also by size exclusion chromatography. Biological relevance of tau-oligomers is shown by preliminary studies using CSF (FIG. 6). These result show that this approach can also be used for testing candidate drug efficacy ex vivo and in vivo. This method of generating and measuring tau oligomers facilitates simple, sensitive high throughput screening to identify compounds that can inhibit this pathological process. The reaction can be performed at neutral pH at 37° C., thus, eliminating the effects of high temperature or pH on the stability of the test compounds. The tau oligomers are amenable for isolation and purification by gel filtration, thus providing a tool to screen and identify compounds that can disrupt oligomer structure.

This simple, sensitive in vitro system facilitates conversion of an assay to high throughput screening (HTS) of compound libraries to identify compounds that inhibit oligomer formation. This method has been standardized to isolate oligomers by size exclusion chromatography that can be used to provide oligomer substrate to screen for compounds that disrupt oligomer structure.

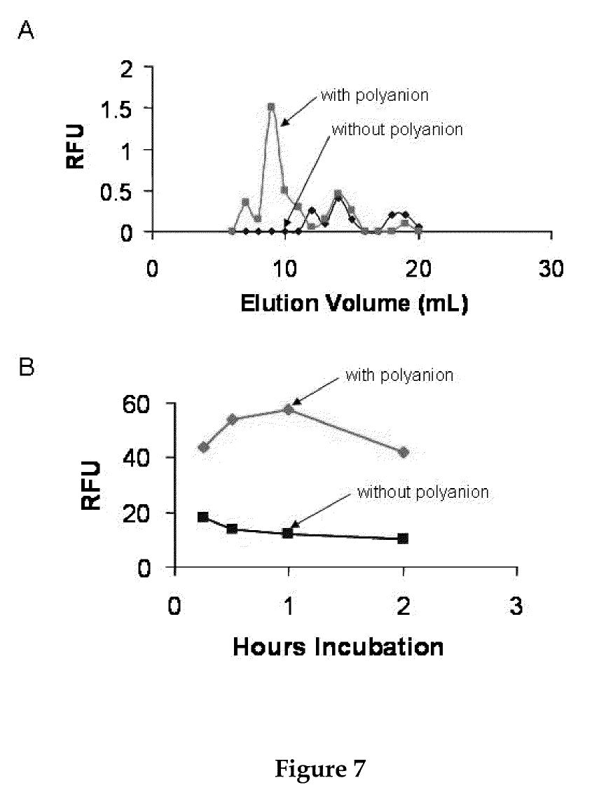

The elution profile of tau412 incubated with RNA and tau 412 incubated without the RNA from Sepahcryl S-300 column is shown in FIG. 7. Tau 412 incubated with RNA facilitator formed high molecular weight peaks predominantly at 680 kDa, and a small peak at 730 kDa, whereas, tau 412 incubated without RNA did not form these high molecular weight species, thus, indicating that RNA facilitated the oligomerization of tau 412.

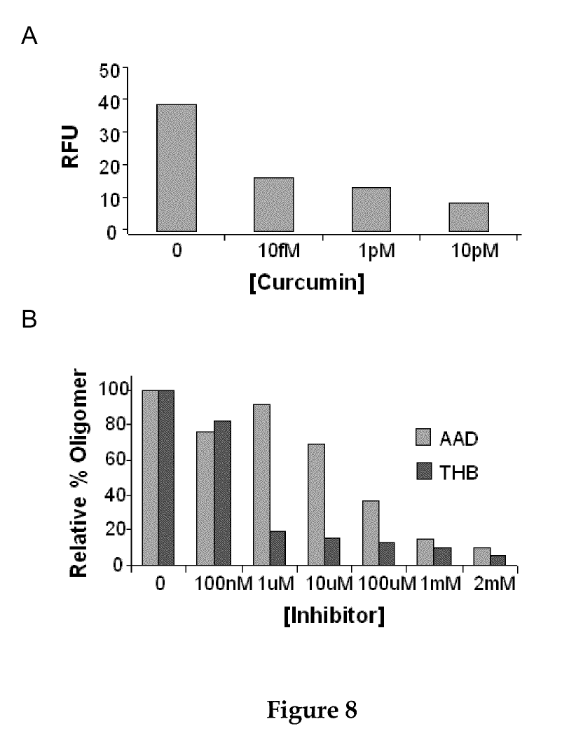

Tau oligomer formation assays was performed with increasing concentrations of curcumin, and oligomers were detected by ELISA (A). 50% inhibition oligomer formation was achieved at ˜10 fM curcumin. B). Tau oligomer formation, resolved by SDS-PAGE, was used to test tool compounds and screen a compound library. Shown on FIG. 8 are results for tool compound 2,3,4-Trihydroxybenzophenone (THB) (maroon bars), and Amino 1,1′-azobenzene-3,4′-disulfonic (AAD) (blue bars).

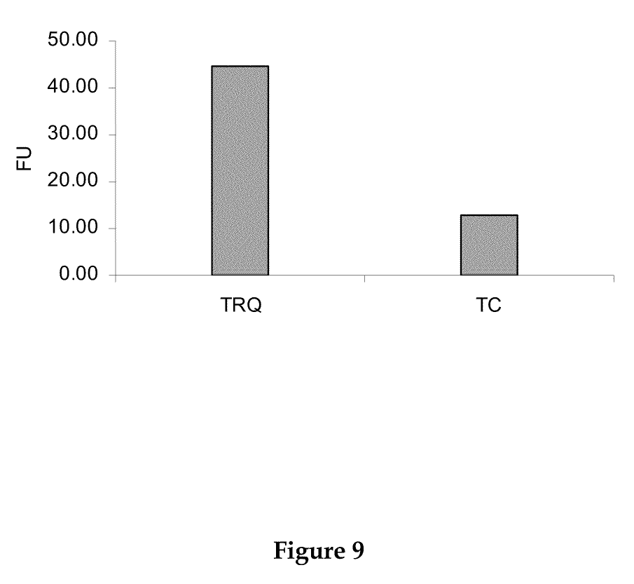

Tau oligomers generated using RNA facilitators show reactivity with polyclonal antibody A11 (FIG. 9), specific for protein oligomer structure (Kayed et al., 2003) in an amplified ELISA.

### Example 4

### Over-Expression of Tau Causes the Formation of Tau Oligomers in Association with Loss of Memory in Mouse Models

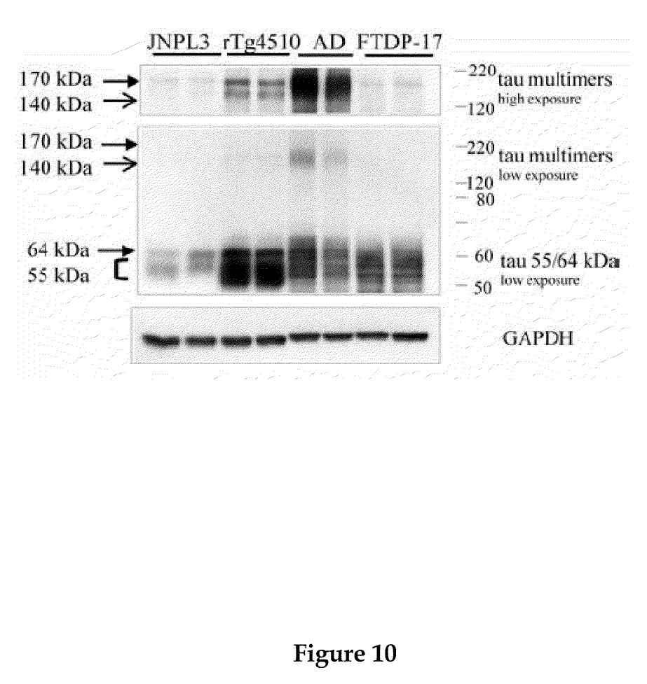

Biochemical analyses performed on brain tissues from mouse models to find tau structures whose levels correlated with memory loss. Two forms of tau multimers (140 and 170 kDa) were found in a mouse model, whose molecular weight indicates an oligomeric aggregate, accumulated early in pathogenesis (FIG. 10). Similar tau multimers were detected in another mouse model of tauopathy and in brain specimens from patients with AD and FTDP-17. Moreover, levels of the tau multimers correlated consistently with memory loss at various ages in the conditional tau P301L mouse model. These findings show that accumulation of early-stage aggregated tau species, before the formation of NFTs, is associated with the development of functional deficits during the pathogenic progression of tauopathy (Berger et al. 2007).

### Example 5

### Chemical Synthesis of 150 Bi-Aromatic Compounds

A library of 195 bi-aromatic compounds has been synthesized and evaluated for activity against inhibition of tau aggregation in Thioflavin S (ThS) dye-binding fluorescence assays. Multiple compounds were active at inhibiting tau self-assembly at compound-to-protein ratios as low as 1:1 Inhibition of tau aggregation was confirmed using SDS-PAGE, in which compounds were found to inhibit tau oligomerization at concentrations of 10 to 100 μM.

Pharmacokinetic (PK) testing in mice has been performed for a small number of representative prototype bi-indole compounds and is underway for several others. The toxicity profile, half-life (t_{1/2}), blood-brain barrier permeability and bioavailability information obtained was positive; compounds administered to mice (IP or PO dosing) had no toxicity at doses up to 300 mg/kg and were present in brain more than four hours after administration.

From the in vitro testing, four lead compounds have been identified for advancement to transgenic animal studies (compounds QR-0109, QR-0161, QR-0194 and QR-0212) as shown in Table 4. In vitro activities and PK data for each compound are summarized herein. As new data on the anti-amyloidogenic activity and PK profile of existing and newly synthesized compounds is continually collected, selection of leads can be frequently re-evaluated and occasionally altered. Thus, the four most promising compounds at initiation of transgenic animal testing studies can be the ones advanced.

TABLE 4
Preliminary lead compounds for present study

| QR-0109 | QR-0161 |
|---|---|
| Inhibits tau aggregation (ThS) | Inhibits tau aggregation (ThS) |
| Non-toxic up to 300 mg/kg, crosses | Non-toxic in mice up to 300 mg/kg |
| blood-brain barrier, has t_{1/2} of several | |
| hours in mouse PK studies | |
| QR-0194 | QR-0212 |
| Inhibits α-synuclein aggregation | Inhibits tau aggregation (ThS) |
| (ThT) | |
| Non-toxic in mice up to 300 mg/kg | Non-toxic in mice up to 300 mg/kg |

### Example 6

### Tau is Also Essential to Aβ Neurotoxicity

The bi-aromatic compounds can be tested in a transgenic Aβ animal model of AD, for example the APP/PSEN1 mouse model in which mice develop Aβ but not tau pathology. This approach can be used to identify compounds acting through an anti-Aβ mechanism. To determine the efficacy of compounds against tau oligomerization and neurotoxicity resulting therefrom, the compounds can be examined in a tauopathy transgenic model, for example, the tau P301L mouse. This approach can be used to characterize the biological activity of the bi-aromatic compounds. Together, the two dementia models can be used to identify the independent contribution of anti-Aβ and anti-tau activities of the compounds, thus providing mechanistic insight for directing further drug optimization efforts.

Validation of tau protein as a drug discovery target for AD is supported by experiments in a mouse model for AD showing that reduction of tau levels protected mice from A13-induced cognitive impairments (FIG. 11; see Roberson et al. 2007; see Boston Globe Article, Appendix III; See Science article, “A New Take on Tau”). Tau reduction also protected both transgenic and nontransgenic mice against excitotoxicity. The results also showed that tau reduction can block Aβ- and excitotoxin-induced neuronal dysfunction and can be an effective strategy for treating AD and related conditions. (For a very recent review of tau-mediated neurodegeneration in AD and related disorders see Ballatore et al. 2007).

Tau oligomers can be formed in vitro and used to screen compounds inhibiting this process. This technology is also valuable in measuring tau oligomers in clinical specimens. These technologies can be used to identify the subset of compounds inhibiting formation of soluble oligomers of tau from a library of compounds already shown to inhibit the formation of insoluble aggregates of Aβ, tau, and α-synuclein and to determine their efficacy in vivo.

Regardless of the size of Aβ, tau and α-synuclein aggregates that effect toxicity, binding to and altering the distribution of aggregates, is can disrupt their toxic action on neurons. This can happen in a number of different ways: 1) compounds can stabilize monomers or aggregates smaller than the one(s) that induce neurotoxicity, thereby reducing the pool of toxic aggregates; 2) compounds can bind to toxic aggregates and block their detrimental interaction at neurons; 3) in binding to toxic aggregates, compounds can promote their breakdown into smaller, non-toxic aggregates; 4) metabolism/clearance of Aβ can be facilitated by compounds which promote a shift to smaller aggregate sizes. Anti-Aβ drug candidates which function through one or more of these pathways include tramiprosate (Kisilvesky, Szarek & Weaver, 1997-2005; Gervais et al., 2006), and isomers of inositol (McLaurin et al., 2006).

This approach can be used to discover and optimize drugs for the treatment of AD. This drug discovery goal can be addressed by optimizing and advancing into animal models recently-developed small organic molecules (“bi-aromatics”) capable of binding to and modulating the aggregation of all three amyloidogenic proteins implicated in AD, i.e. Aβ, tau and α-synuclein (Carter et al., U.S. application Ser. No. 11/443,396, 2006). Unlike tramiprosate (Gervais et al., 2006) and inositol (McLaurin et al., 2006), which have only been may work against Aβ, the bi-aromatics can benefit from synergism, i.e. by acting at three different targets in AD, the net effect of the compounds can be greater than the sum of the individual effects. The compounds selected in this program can have the additional advantages of pre-clincal screening for effects on synaptic plasticity ex-vivo and behavioural deficits in vivo, unlike tramiprosate.

### Example 7

### Screening of Compounds for Inhibition of Tau Soluble Oligomer Formation

For inhibition of tau oligomer formation analysis, RNA can be used to facilitate tau oligomer formation in the presence of test compounds at various concentrations, and oligomers formed can be measured by ELISA. For tau oligomer formation 20 pmol tau412 and 4 pmol of the RNA facilitator can be incubated in 50 ul 50 mM Tris HCl pH 7.4 for 1 hr at 37° C. A negative control can be run in parallel in which the RNA is replaced with buffer. Compounds can be tested at a range of concentrations from 1 nM to 1 mM using oligomer formation conditions. Each sample can be tested in triplicate. The four lead compounds (QR-0109, QR-0161, QR-0194 and QR-0212) can be tested first and additional compounds can be tested as they are synthesized and delivered. ELISA for measuring tau oligomers can use capture antibody MN1000 (Pierce) against tau, reporter antibody All (Invitrogen), specific for oligomer conformation, biotinylated anti-rabbit IgG, and neutravidin-HRP with Quanta-blu substrate (Pierce) for fluorogenic detection using a Fluoroskan II fluorometer (excitation 325 nm; emission 420 nm). An ELISA using mAbMN1000 for both capture and reporter can be used to measure soluble aggregates of tau to corroborate results of the All ELISA. An assay measuring the formation of tau oligomers stable in buffer containing SDS and β-mercaptoethanol after 48 hr incubation at 37° C. can be performed using SDS PAGE for additional data on the effect of the compounds on tau-tau interactions (see Neuroscience 2006 poster, appended).

### Example 8

### Screening Test Compounds in P310L Mice

After test compounds are screened in hippocampal slices treated with tau oligomers to determine whether they are capable of re-establishing normal synaptic function, the compounds can be tested in hemizygous tau P301L (Taconic) mice for rescue of behavioral abnormalities. Brain and CSF samples from the transgenic mice can be evaluated for levels of tau oligomers to determine if there is a correlation between compound efficacy and tau oligomer burden.

Both WT mice and hemizygous tau P301L mice can be purchased from Taconic (Lewis et al, 2000). Electrophysiological Analysis can be performed on males (see detailed description in reference (Gong et al., 2006), 400 p.m slices can be cut with a tissue chopper and maintained in an interface chamber at 29° C. for 90 min prior to recording. Briefly, CA1 fEPSPs can be recorded by placing both the stimulating and the recording electrodes in CA1 stratum radiatum. Basal synaptic transmission (BST) can be assayed by plotting the stimulus voltages against slopes of fEPSP, or by plotting the peak amplitude of the fiber volley against the slope of the fEPSP. For LTP experiments, a 15 minute baseline can be recorded every min at an intensity that evokes a response ˜35% of the maximum evoked response. LTP can be induced using θ-burst stimulation (4 pulses at 100 Hz, with the bursts repeated at 5 Hz and each tetanus including 3 ten-burst trains separated by 15 sec).

All the experiments can be performed in blind. Results can be expressed as Standard Error Mean (SEM). Level of significance can be set for p<0.05. Results can be analyzed with ANOVA with post-hoc correction.

### Example 9

### Determining if Treatment with Compounds has Beneficial Effect on Behavioral Abnormalities in Tau P301L Mice

Concomitant inhibitors of Aβ, tau and α-synuclein aggregation can rescue the behavioral deficits observed in Tau P301L mice of 6 months of age and treatment with the aggregation inhibitors can have beneficial effects on abnormal behavior in Tau P301L mice.

Behavioral testing can be performed on compounds indentified with any in-vitro screens described herein. Conditions that can be tested include: hemizygous Tau P301L and WT treated with aggregation inhibitors, hemizygous Tau P301L and WT treated with vehicle. The treatment can be started at 5 months of age. Mice can be tested at 6 months of age. Motor function can be monitored twice a week by wire hang, and beam walk tests and then once a day when the first signs of motor impairments became evident (Le Corre, et al, 2006). Animals showing at least two consecutive hang-test failures for two consecutive days and or drops from the beam two consecutive times for two consecutive days and or animals that fail once at the hang test and simultaneously fall once from the beam for two consecutive days can be rated as having severe motor deficits and can be killed. The remaining mice that appear unaffected throughout the whole study period or develop mild motor abnormalities below formal threshold criteria for removal can be killed at the end of the study period,their blood and brains used for measurement of Tau oligomer levels. As a control for effectiveness of aggregation inhibitors, hippocampal compound levels in Tau P301L mice can be measured after administration of the compounds. If the compounds have a beneficial effect, there may be no difference or little difference in the motor tasks between compound-treated Tgs and WT littermates, whereas vehicle-treated Tgs may show abnormal learning behavioral performance. Compound-treated WT mice may show normal performance. If so, these results can indicate that treatment with these compounds can prevent the development of behavioral abnormalities in the tauopathy model.

If treatment with the compound at 6 months (when tangles have already started to form) is not sufficient to rescue behavioral impairments in the Tau P301L mice (whose mutated transgenes are expressed for an extended period of time), drug administration can be started before tangle appearance (4 months) and prolonged it until the day of behavioral experiments (6 months).

If the compounds have long-term toxicity that can not be detected in acute pharmacokinetic/toxicity testing, attrition of the mouse population over the course of the study can occur due to toxicity/side-effects. Although Tau P301L mice reproduce some aspects of AD, they do not reproduce the disease. At this point in time no animal model for tauopathy fully recapitulates the natural course of the disease, though several Tg mice have been developed that exhibit components of the pathology. The ultimate test both in the studies described herein and in other researches utilizing Tg animals can be done on individuals affected by the disease.

Experiments can be performed in blind both on male and female animals (sex can be balanced across experiments). Mice can be videotaped for 15 sec. The video can be played back in half time for analyses. The wire hang test can measure the latency to fall from an inverted grid. For the beam walk test animals can be trained to traverse a 130 cm long beam from a platform at one end to the animal's home cage to the other end, and the number of foot slips onto an under-hanging ledge can be recorded.

For all experiments mice can be coded to blind investigators with respect to genotype and treatment. Results can be expressed as Standard Error Mean (SEM). Level of significance can be set for p<0.05. Results can be analyzed with ANOVA with post-hoc correction with drug or genotype as main effect. Experiments can be designed in a balanced fashion, and mice can be trained and tested at each of the different conditions in 3 or 4 separate experiments.

### Example 10

### Reactive and Unreactive Tau Oligomers

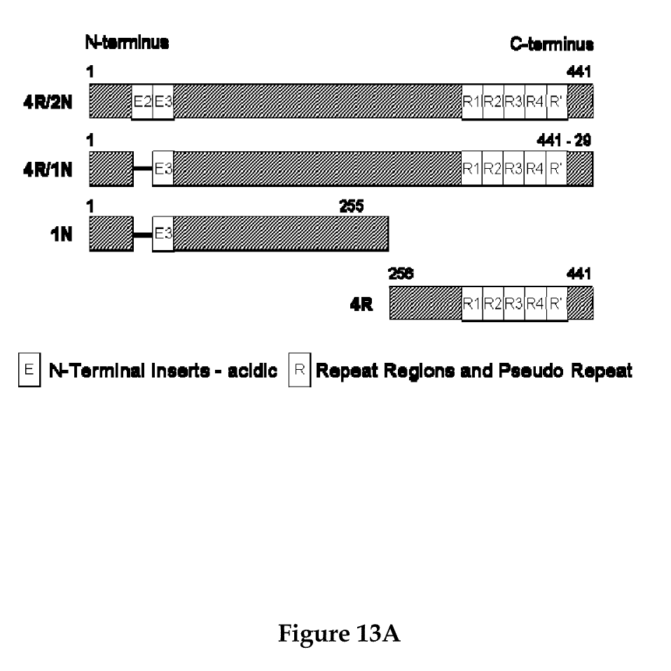

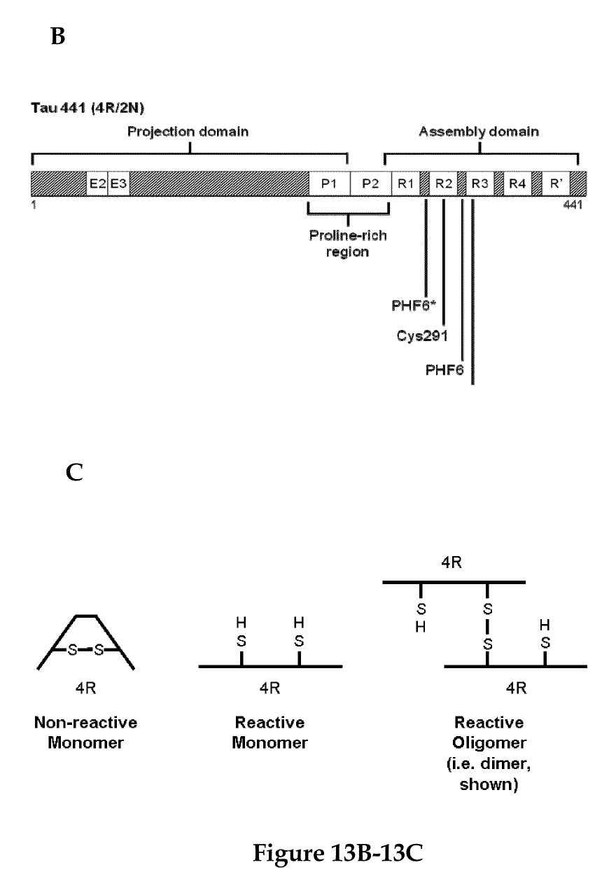

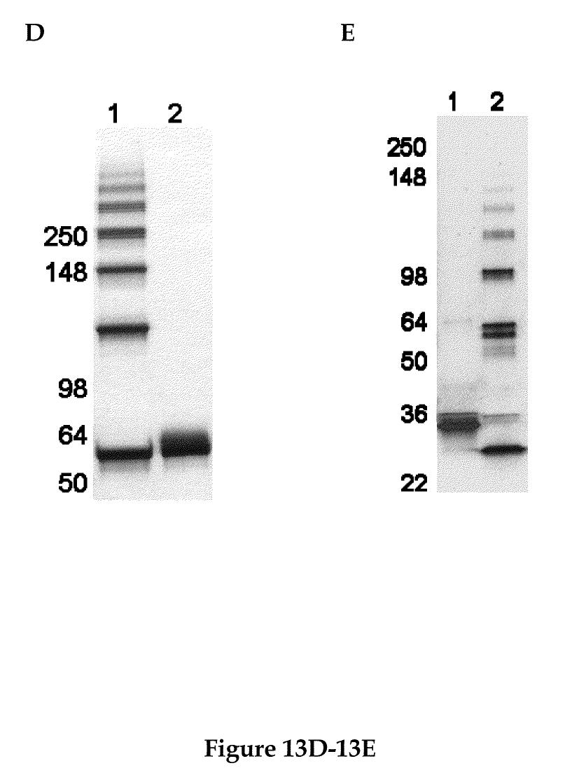

Tau isoforms containing four microtubule binding repeats and either one or two N-terminal inserts (4R/1N, 4R/2N) (FIG. 13A) are often associated with neurodegenerative diseases (Goedert and Jakes 2005). The physiological concentrations of extracellular tau vary from about 200-400 pg/ml (4-8 pM) in normal CSF to about 400-1000 pg/ml (8-20 pM) in AD CSF (Iqbal et al. 2005). SDS-stable tau oligomers have been detected in the cortex of AD brain by immunoblot (Berger et al. 2007; Maeda et al. 2007), and granular tau oligomers were found to accumulate in AD cortex using atomic force microscopy (Maeda et al. 2006; 2007). To determine the relevance of tau oligomers in synaptic plasticity and memory tau monomer and disulfide-mediated tau oligomers were purified from oligomerized preparations of purified tau monomer. Disulfide linkages can form between cysteine residues 291 and 322 in the second and third microtubule binding repeats in the carboxyl-terminal half of tau protein (FIG. 13B). Intermolecular disulfide bond formation leads to formation of disulfide-mediated tau oligomers, whereas intramolecular disulfide bond formation prevents tau oligomer formation. Unreactive tau monomer was tau monomer that did not form disulfide linked oligomers following an oligomerization reaction (FIG. 13C). Unreactive monomer can be induced to oligomerize upon addition of reductant indicating that the unreactive monomer contains an intramolecular disulfide bond precluding it from forming intermolecular disulfide linkages with other tau molecules. The distribution of monomer and oligomer species was about 50% monomer, 25% dimer, and 25% higher order oligomers (FIG. 13D). The N-terminal construct did not form oligomers, whereas the C-terminal tau 4R construct oligomerized (FIG. 13E), supporting the observation that the microtubule binding repeats are responsible for tau oligomerization (von Bergen et al. 2000).

### Example 11

### Extracellular Tau Oligomers Impair LTP

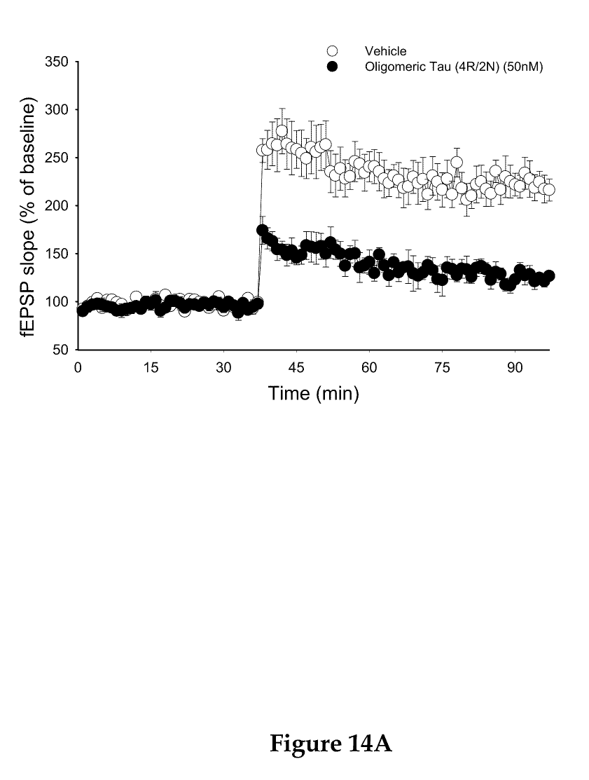

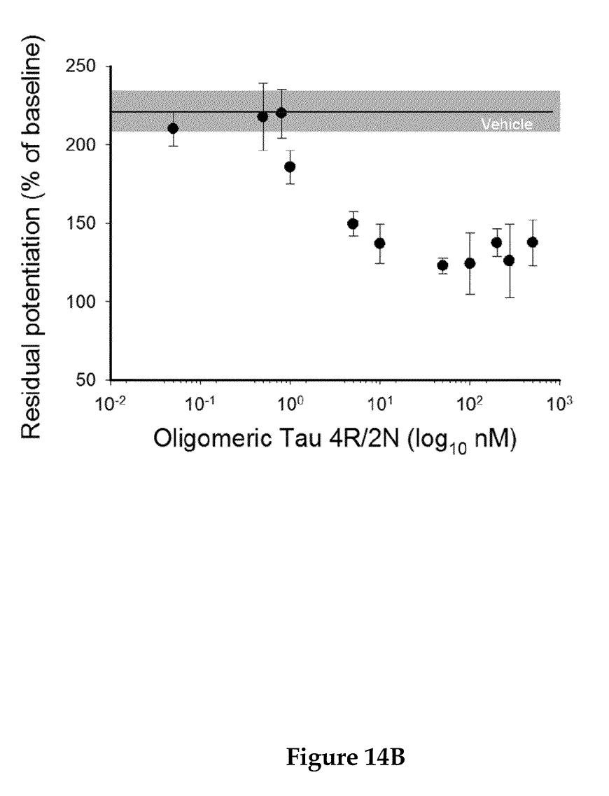

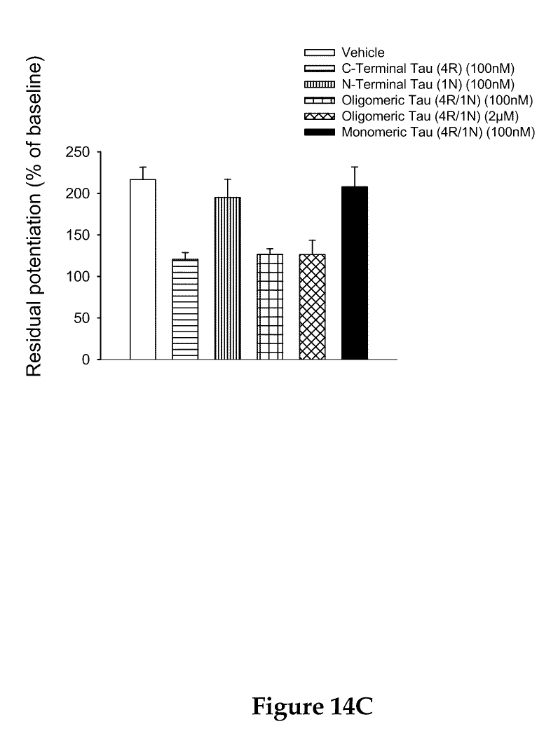

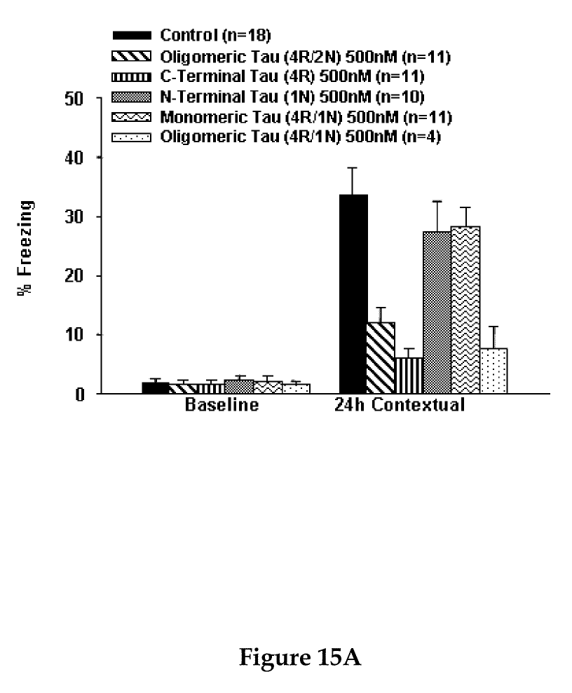

Long term potentiation (LTP), an electrophysiological correlate of learning and memory, was examined to determine whether a brief application of tau oligomers was capable of producing a defect in potentiation at the connection between Schaffer collateral and CA1 pyramidal neurons in the hippocampus. Hippocampal slices were perfused with a solution containing oligomer-enriched 4R/2N tau for 20 min before inducing LTP through tetanic stimulation of the Schaffer collateral pathway. Application of 50 nM tau did not affect basal transmission as determined by the stable baseline both before and after tau application. Theta-burst stimulation, in turn, markedly reduced LTP (FIG. 14A). To verify and further characterize the inhibition of LTP by extracellular tau oligomers a dose-response curve was generated, the IC₅₀ was determined to be 4.3 nM, and the maximal effect was observed at 50 nM oligomeric tau preparation (FIG. 14B). Similar reductions of potentiation were obtained when different concentrations (100 nM and 2 μM) of a preparation containing oligomeric 4R/1N tau were used, indicating that the number of N-terminal inserts is not critical for the reduction of LTP (FIG. 14C).

To determine which domain of tau contributes to synaptic dysfunction, the N- and C-terminal portions of tau were tested using the LTP assay. The oligomerized C-terminal construct (100 nM) reduced LTP, whereas the N-terminal construct (100 nM), which did not oligomerize, did not reduce potentiation. Moreover, the purified unreactive 4R/1N monomer did not affect LTP (FIG. 14C), indicating that inhibition of synaptic plasticity by tau is dependent on its oligomerization.

### Example 12

### Extracellular Tau Oligomers Impair LTP

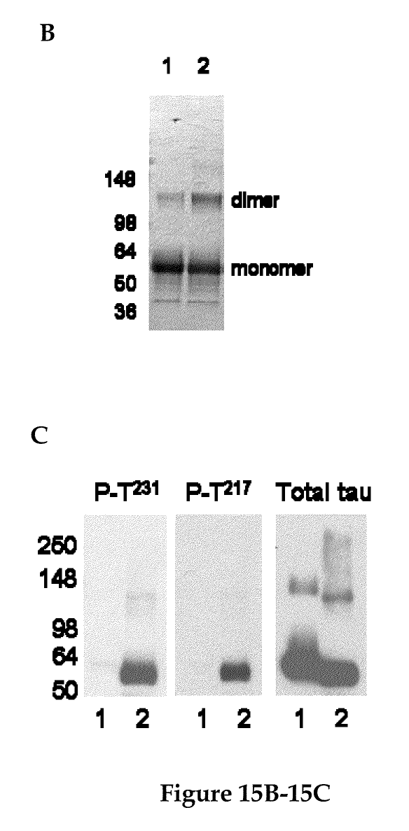

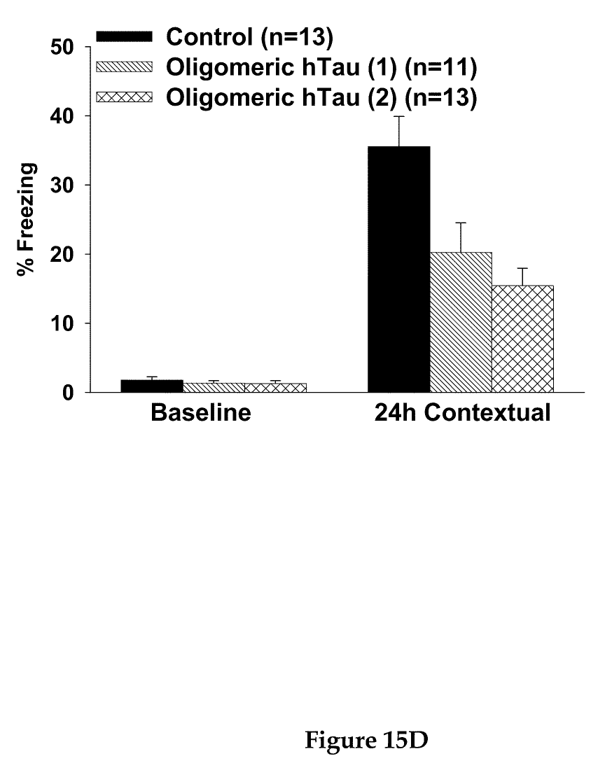

Given that LTP is thought to underlie learning and memory, the effects of extracellular tau was evaluated using the contextual fear conditioning model of associative learning, a type of memory that is impaired in AD patients (Swainson et al, 2001) and depends upon hippocampal and amygdala function (Phillips and LeDoux 1992). Cannulas were implanted bilaterally into the mouse dorsal hippocampi. After 5-7 days, animals were trained to associate neutral stimuli with an aversive one. They were infused with oligomeric tau 4R/2N (500 nM), oligomeric 4R/1N (500 nM), oligomeric C-terminal tau 4R (500 nM), monomeric tau 4R/1N (500 nM) and monomeric N-terminal tau 1N (500 nM) in a final volume of 1 μl over 1 min or vehicle (two injections at 180 min and 20 min prior to applying the electric shock). No difference was found in the freezing behavior among the groups of mice during the training phase of the fear conditioning. Memory was assessed 24 hours later by measuring freezing behavior, the absence of all movement except for that necessitated by breathing, in response to representation of the context. Mice treated with oligomeric tau 4R/2N, tau 4R/1N, or C-terminal tau 4R showed a reduction of freezing, whereas N-terminal tau 1N treated mice or mice treated with monomeric tau 4R/1N showed normal freezing (FIG. 15A). When cued fear learning, a type of learning that depends upon amygdala function (Phillips and LeDoux, 1992), was assessed on the same animals twenty-four hours after contextual learning by measuring freezing in response to representation of the auditory cue within a completely different context, no difference between tau-infused mice and vehicle-infused mice was observed, indicating that behavioral changes produced by tau were due to a selective hippocampus-dependent effect on fear learning. These experiments indicate that extracellular tau oligomers impair associative fear memory.

The effect of oligomeric tau purified from human AD brain specimens with tau pathology was examined (FIG. 15B). Two methods were used to increase the oligomer content in the preparation. The first method used a 20 min incubation at 90° C. with 5 mM DTT, and in the second method the preparation was concentrated and buffer exchanged into 50 mM Tris-HCl pH 7.4 and incubated at 60° C. for 1 hr to increase the amount of tau oligomers (FIG. 15B). The second method led to the formation of more tau oligomer content than the first method. For all experiments the tau preparations were buffer exchanged into artificial cerebrospinal fluid (ACSF) buffer prior to use Immunoblot analysis showed that the tau was phosphorylated at tyrosine residues 217 and 231 indicating that tau in the preparation maintained its phosphorylated status (FIG. 15C). Mice were injected with oligomeric human AD tau (500 nM) or vehicle in a final volume of 1 μl over 1 min or vehicle, 180 and 20 minutes prior to applying the electric shock, and tested for contextual fear memory. No difference was found in the freezing behavior among the groups of mice during the training phase of the fear conditioning. Contextual fear learning, in turn, was reduced in mice treated with oligomeric human AD tau (FIG. 15D). Amygdala-dependent cued learning was the same among the different groups of mice. These experiments demonstrate that the recombinant tau oligomers are a good representation of AD tau oligomers and that tau oligomerization, rather than post-translational modifications of the AD tau, is required for reduction in associative memory.

### Example 13

### Tau Protein Preparation

The cDNA for tau412 was purchased from OriGene and subcloned into the bacterial expression vector pET21B to produce the tau protein. Similarly, the amino and carboxyl-terminal tau fragments were subcloned from this cDNA. The tau 441 construct was purchased from Bioclone ready to express in bacteria. The bacterial strain BL21(DE3) was used for protein expression. Standard protocols from the cell and vector distributor (Novagen) were used to grow the cells and express the protein. Briefly, cells were streaked on LB agar ampicillin plates and a single colony was picked and grown overnight in 2 ml 2XYT medium with glucose and 100 mg/ml carbenicillin. The overnight culture was used to inoculate a 500 ml culture which was grown to an optical density of 0.8 U/ml at 600 nm wavelength. Protein expression was induced with 1 mM IPTG for 4 hours at which time cells were pelleted at 4° C. by centrifugation at 6000 g. Pellets were stored overnight at −80° C. Cell lysates were prepared with Cell Lytic B lysis buffer, lysozyme, benzonase, and protease inhibitors according to manufacturer's protocol (Sigma). Cation exchange resin SP-Sepharose was used for the first step of purification and 300 mM NaCl was used to elute tau protein. The second purification step used His-bind resin to bind the his-tagged tau protein which was eluted with imidazole. Buffer exchange into 50 mM Tris-HCl pH 7.4 was performed with Amicon Ultra Centrifugal Devices. Protein concentration was determined with BCA assay. Tau oligomers were generated by incubation of 5 μM tau in 50 mM Tris buffer pH 7.4 with 100 mM NaCl at 37° C.

### Example 14

### Methods for Isolating Tau Form AD Brain Tissue and Forming Oligomers

Tau was purified from brain tissue of a 44-year-old male with tau pathology characterized by numerous argyrophilic neuronal tangles (Bielschowsky), and innumerable AT8-labeled neurons throughout the cerebral cortex including the motor cortex, and visual cortex. Tissue was acquired from the New York Brain Bank—The Taub Institute, Columbia University. The method described by Ivanovova et al. (2008) was generally followed, but fractionation based on size and charge was used in place of the immunoaffinity column for the final purification step. An advantage of this method is that tau phosphorylation is preserved using homogenization buffer containing 1% perchloric acid. Reductant was used during purification and fractions containing monomeric tau were pooled, concentrated and buffer exchanged into 50 mM Tris-HCl pH 7.4. Two methods were used to increase the oligomer content in the preparation. The first method used a 20 min incubation at 90° C. with 5 mM DTT. In the second method the preparation was concentrated and buffer exchanged into 50 mM Tris-HCl pH 7.4 and incubated at 60° C. for 1 hr to increase the amount of tau oligomers (FIG. 15B).

### Example 15

### Methods for AD Tau Immunoblot

50 ng recombinant tau441 and AD tau were run on precast 4-20% gradient polyacrylamide gels (BioRad) and transferred to PVDF membrane (Millipore) Immunoblots for total tau were performed with monoclonal antibody HT7 (Pierce/Thermo Scientific), and with phosphospecific polyclonal antibodies against tau [pT217] and [pT231] (Invitrogen). Duplicate blots were first performed with the phospho-tau antibodies and reused for total tau analysis.

### Example 16

### Animals

3-4 month-old male C57BL/6J mice) were obtained from a breeding colony kept in the animal facility at Columbia University. All mice were maintained on a 12 h light/dark cycle (with lights on at 6:00 A.M.) in temperature- and humidity-controlled rooms of the Columbia University animal facility.

### Example 17

### Electrophysiological Studies

Transverse hippocampal slices (400 μm) were cut with a tissue chopper and maintained in an interface chamber at 29° C. for 90 min prior to recording, as previously described (Puzzo et al, 2008). The extracellular artificial cerebrospinal fluid consisted of 124.0 mM NaCl, 4.4 mM KCl, 1.0 mM Na₂HPO₄, 25.0 mM NaHCO₃, 2.0 mM CaCl₂. 2.0 mM MgSO₄, and 10.0 mM glucose, continuously bubbled with 95% O₂/5% CO₂ to a final pH of 7.4. Field extracellular postsynaptic potentials (fEPSPs) were recorded by placing the stimulating and recording electrodes in CA1 stratum radiatum. A bipolar tungsten electrode (FHC, Bowdoin, Me.) was used as a stimulating electrode, and a glass pipette filled with bath solution was used as a recording electrode. Following assessment of basal synaptic transmission by plotting the stimulus voltages against slopes of fEPSP, baseline was recorded every minute at an intensity that evoked a response 35% of the maximum evoked response. Slices were perfused for 20 min with different tau preparations or vehicle, and LTP was induced using a theta-burst stimulation (4 pulses at 100 Hz, with the bursts repeated at 5 Hz and each tetanus including 3 ten-burst trains separated by 15 sec). Responses were measured as fEPSP slopes expressed as percentage of baseline.

### Example 18

### Behavioral Studies

Cannula infusion technique. Cannulas were implanted onto both dorsal hippocampi following anaesthesia with 20 mg/kg Avertin, as previously described (Puzzo et al, 2008). They were fixed to the skull with acrylic dental cement (made from Paladur powder). After 5-7 days, mice were bilaterally infused with tau preparations or vehicle in a final volume of 1 μl over 1 min at 180 and 20 minutes prior to the foot shock, with a microsyringe connected to the cannulas via polyethylene tubing. Mice were handled once a day for 3 days before behavioral assessment. During infusion animals were handled gently to minimize stress. After infusion, the needle was left in place for another minute to allow diffusion. After behavioral testing, a solution of 4% methylene blue was infused into the cannulas to check for the position of the cannulas, as previously described (Puzzo et al, 2008).

Fear conditioning (FC) was assessed as previously described (Puzzo et al, 2008). Mice were placed in a conditioning chamber for 2 min before the onset of a tone (Conditioning Stimulus, CS) (a 30 sec, 85 dB sound at 2800 Hz). In the last 2 sec of the CS, mice were given a 2 sec, 0.6 mA foot shock (Unconditioning Stimulus, US) through the bars of the grid-floor and left in the conditioning chamber for additional 30 sec. Freezing behavior (the absence of all movements except for those needed for breathing) was scored using a Freezeview software. Contextual fear learning was evaluated 24 hrs after training by measuring freezing for 5 min in the chamber in which mice had been trained. Cued fear learning, a type of memory depending upon amygdala function (Phillipps and LeDoux, 1992), was assessed 24 hrs after contextual testing by placing mice in a novel context for 2 min (pre-CS test), after which they were exposed to the CS for 3 min (CS test). To determine whether the treatments with tau preparations affected sensory perception of the mice, threshold assessment was conducted as previously described (Puzzo et al, 2008). Briefly, in the threshold assessment test the animals were placed in the conditioning chamber and the electric current (0.1 mA for 1 sec) was increased at 30 sec intervals from 0.1 mA to 0.7 mA. Threshold to flinching (first visible response to shock), jumping (first extreme motor response), and vocalized response were quantified for each animal by averaging the shock intensity at which each the animal showed the behavioral response to that type of shock. Different tau preparations did not affect sensory threshold.

### Example 19

### Statistical Analyses

Experiments were performed in blind. Results were expressed mean±the standard error of the mean (SEM) (level of significance at p<0.05). Results were analyzed by the Student's t test (paired comparisons) or ANOVA for repeated measures (multiple comparisons) with treatment condition as main effect. Planned comparisons were used for post-hoc analysis. Bbehavioral experiments were designed in a balanced fashion. For each condition, mice were trained and tested in three to four separate sets of experiments.

### REFERENCES

Adler V, Davidowitz EJ, Tamburi P, Rojas P and Grossman A. 2-Macroglobulin is a potential facilitator of prion protein transformation. Amyloid, January 2007, In Press.
Arancio, O. and A. B. MacDermott, Differential distribution of excitatory amino acid receptors on embryonic rat spinal cord neurons in culture. J Neurophysiol, 1991. 65(4): p. 899-913.
Arancio, O., E. R. Kandel, and R. D. Hawkins, Activity-dependent long-term enhancement of transmitter release by presynaptic 3′,5′-cyclic GMP in cultured hippocampal neurons. Nature, 1995. 376(6535): p. 74-80.
Arancio, O., H. Korn, A. Gulyas, T. Freund, and R. Miles, Excitatory synaptic connections onto rat hippocampal inhibitory cells may involve a single transmitter release site. J Physiol, 1994. 481 (Pt 2): p. 395-405.
Arancio, O., M. Kiebler, C. J. Lee, V. Lev-Ram, R. Y. Tsien, E. R. Kandel, and R. D. Hawkins, Nitric oxide acts directly in the presynaptic neuron to produce long-term potentiation in cultured hippocampal neurons. Cell, 1996. 87(6): p. 1025-35.
Arendash, G. W., D. L. King, M. N. Gordon, D. Morgan, J. M. Hatcher, C. E. Hope, and D. M. Diamond, Progressive, age-related behavioral impairments in transgenic mice carrying both mutant amyloid precursor protein and presenilin-1 transgenes. Brain Res, 2001. 891(1-2): p. 42-53.
Asuni A A, Boutajangout A, Quartermain D, Sigurdsson E M Immunotherapy targeting pathological tau conformers in a tangle mouse model reduces brain pathology with associated functional improvements. J Neurosci. 2007 Aug. 22; 27(34):9115-29.
Ballatore C, Lee V M, Trojanowski J Q. Tau-mediated neurodegeneration in Alzheimer's disease and related disorders. Nat Rev Neurosci. 2007 September; 8(9):663-672.
Bartus R T, Dean R L I, Beer B, Lippa A S. The cholinergic hypothesis of geriatric memory dysfunction. Science 1982. 217: 408-417.
Berger Z, Roder H, Hanna A, Carlson A, Rangachari V, Yue M, Wszolek Z, Ashe K, Knight J, Dickson D, Andorfer C, Rosenberry T L, Lewis J, Hutton M, Janus C. Accumulation of pathological tau species and memory loss in a conditional model of tauopathy. J Neurosci. 2007 Apr. 4; 27(14):3650-62.
Borchelt, D. R., T. Ratovitski, J. van Lare, M. K. Lee, V. Gonzales, N. A. Jenkins, N. G. Copeland, D. L. Price, and S. S. Sisodia, Accelerated amyloid deposition in the brains of transgenic mice coexpressing mutant presenilin 1 and amyloid precursor proteins. Neuron, 1997. 19(4): p. 939-45.
Chalifour R J, McLaughlin R W, Lavoie L, Morissette C, Tremblay N, Boule M, Sarazin P, Stea D, Lacombe D, Tremblay P, Gervais F. Stereoselective interactions of peptide inhibitors with the beta-amyloid peptide. J Biol Chem 2003. 278: 34874-81.
Chapman, P. F., G. L. White, M. W. Jones, D. Cooper-Blacketer, V. J. Marshall, M. Irizarry, L. Younkin, M. A. Good, T. V. Bliss, B. T. Hyman, S. G. Younkin, and K. K. Hsiao, Impaired synaptic plasticity and learning in aged amyloid precursor protein transgenic mice. Nat Neurosci, 1999. 2(3): p. 271-6.
Cheng I H, Scearce-Levie K, Legleiter J, Palop J J, Gerstein H, Bien-Ly N, Puolivali J, Lesne S, Ashe K H, Muchowski P J, Mucke L. Accelerating amyloid-beta fibrillization reduces oligomer levels and functional deficits in Alzheimer disease mouse models. J Biol. Chem. 2007 282(33):23818-23828.
Davidowitz E, Dover K, Faynshteyn I, Grossman A I, Kisselev S, Moe J G, Rojas P M & Zerlin M. RNA chaperone induced transformation of Tau protein. 2005. Therapeutic Strategies against Neurological Conditions, GTCbio, Boston. Poster
Di Rosa, G., T. Odrijin, R. A. Nixon, and O. Arancio, Calpain inhibitors: a treatment for Alzheimer's disease. J Mol Neurosci, 2002. 19(1-2): p. 135-41.
Diamond, D. M., C. R. Park, K. L. Heman, and G. M. Rose, Exposing rats to a predator impairs spatial working memory in the radial arm water maze. Hippocampus, 1999. 9(5): p. 542-52.
Duda J E, Lee V M, Trojanowski J Q. Neuropathology of synuclein aggregates. J Neurosci Res 2000. 61: 121-7.
Duff, K., C. Eckman, C. Zehr, X. Yu, C. M. Prada, J. Perez-tur, M. Hutton, L. Buee, Y. Harigaya, D. Yager, D. Morgan, M. N. Gordon, L. Holcomb, L. Refolo, B. Zenk, J. Hardy, and S. Younkin, Increased amyloid-beta42(43) in brains of mice expressing mutant presenilin 1. Nature, 1996. 383(6602): p. 710-3.
Frasier M, Walzer M, McCarthy L, Magnunson D, Lee J M, Haas C, Kahle P and Wolozin B. Tau phosphorylation increases in symptomatic mice overexpressing A3OP -synuclein. Exp Neurol 2005. 193: 274-287.
Gervais F, Paquette J, Morissette C, Krzywkowski P, Yu M, Azzi M, Lacombe D, Kong X, Aman A, Laurin J, Szarek W A, Tremblay P. Targeting soluble Abeta peptide with Tramiprosate for the treatment of brain amyloidosis. Neurobiol Aging, online pre-publication, available May 1, 2006. 2007 April; 28(4):537-47.
Goedert M, Jakes R. Mutations causing neurodegenerative tauopathies. Biochim Biophys Acta. 2005, 1739(2-3):240-50.
Gomez-Ramos A, Diaz-Hernandez M, Cuadros R, Hernandez F, Avila J. Extracellular tau is toxic to neuronal cells. FEBS Lett. 2006 Sep. 4; 580(20):4842-50. Epub 2006 Aug. 8.
Gong, B., O. V. Vitolo, F. Trinchese, S. Liu, M. Shelanski, and O. Arancio, Persistent improvement in synaptic and cognitive functions in an Alzheimer mouse model following rolipram treatment. J. Clin. Invest., 2004. 114: p. 1624-1634.
Gong, B., Z. Cao, P. Zheng, O. V. Vitolo, S. Liu, A. Staniszewski, D. Moolman, H. Zhang, M. Shelanski, and O. Arancio, Ubiquitin Hydrolase Uch-Ll Rescues beta-Amyloid-Induced Decreases in Synaptic Function and Contextual Memory. Cell, 2006. 126(4): p. 775-88.
Goryunov D, Liem R K. CHIP-ping away at tau. J Clin Invest. 2007 March; 117(3):590-2.
Grossman A, Savvateeva-Popova E V, Popova A V, Nikitina E A, Medvedeva A V, Peresleni A I, Korochkin L I, Vasan S, Zerlin M, Moe J G, K I Pyatkov, O G Zatsepina, N G Schostak, E S Zelentsova & M B Evgen'ev. Small RNA Chaperones Facilitate Amyloid Protein Transformation in vitro, and Aggregate Formation and Behavioral Abnormalities in Drosophila melanogaster. 2006. Protein Misfolding Diseases: Mechanisms of Misfolding, Pathology and Therapeutic Strategies, Keystone Symposia; Breckenridge, Colo.
Haass C, Selkoe D J. Soluble protein oligomers in neurodegeneration: lessons from the Alzheimer's amyloid beta-peptide. Nat Rev Mol Cell Biol. 2007 February; 8(2):101-12.
Holcomb, L., M. N. Gordon, E. McGowan, X. Yu, S. Benkovic, P. Jantzen, K. Wright, I. Saad, R. Mueller, D. Morgan, S. Sanders, C. Zehr, K. O'Campo, J. Hardy, C. M. Prada, C. Eckman, S. Younkin, K. Hsiao, and K. Duff, Accelerated Alzheimer-type phenotype in transgenic mice carrying both mutant amyloid precursor protein and presenilin 1 transgenes. Nat Med, 1998. 4(1): p. 97-100.
Hsiao, K., P. Chapman, S. Nilsen, C. Eckman, Y. Harigaya, S. Younkin, F. Yang, and G. Cole, Correlative memory deficits, Abeta elevation, and amyloid plaques in transgenic mice. Science, 1996. 274(5284): p. 99-102.
Iqbal K, Flory M, Khatoon S, Soininen H, Pirttila T, Lehtovirta M, Alafuzoff I, Blennow K, Andreasen N, Vanmechelen E, Grundke-Iqbal I. Subgroups of Alzheimer's disease based on cerebrospinal fluid molecular markers. Ann Neurol. 2005, 58(5):748-57.
Ivanovova N, Handzusova M, Hanes J, Kontsekova E, Novak M. High-yield purification of fetal tau preserving its structure and phosphorylation pattern. J Immunol Methods. 2008, 339(1):17-22.
Janus, C., J. Pearson, J. McLaurin, P. M. Mathews, Y. Jiang, S. D. Schmidt, M. A. Chishti, P. Home, D. Heslin, J. French, H.T. Mount, R. A. Nixon, M. Mercken, C. Bergeron, P. E. Fraser, P. St George-Hyslop, and D. Westaway, A beta peptide immunization reduces behavioural impairment and plaques in a model of Alzheimer's disease. Nature, 2000. 408(6815): p. 979-82.
Kaneko I, Yamada N, Sakuraba Y, Kamenosono M, Tutumi S Suppression of mitochondrial succinate dehydrogenase, a primary target of β-amyloid, and its derivative racemized at Ser residue. J Neurochem 1995. 65: 2585-93.
Katsuse, O., Lin, W. L. Lewis, J., Hutton, M. L., Dickson, D. W. Neurofibrillary tangle related synaptic alterations of spinal motor neurons of P301L tau transgenic mice Neuroscience Letters 2006 409: 95-99.
Kayed, R., Thompson, H. E., McIntire T. M., Milton, S. C., Cotman, C. W., and Glabe, C. G. Common structure of soluble amyloid oligomers implies common mechanism of pathogenesis. Science. 2003. 301, 1847-9.
Kirkitadze. M. D., Bitan, G., and Teplow, D. B. Paradigm Shifts in Alzheimer's disease and Other Neurodegenerative Disorders: The Emerging Role of Oligomeric Assemblies. J. Neurosc. Res. 2002. 69: 567-577.
Kisilevsky R, Szarek W, Weaver D. “Method for Treating Amyloidosis”, Japanese patent No. 363236, issued Dec. 3, 2004.
Kisilevsky R, Szarek W, Weaver D. “A Method of Treating Amyloidosis”. U.S. Ser. No. 08/403,230, issued Jul. 1, 1997, U.S. Pat. No. 5,643,562.
Kisilevsky R, Szarek W, Weaver D. “A Method of Treating Amyloidosis”. U.S. Ser. No. 08/472,692, issued Mar. 17, 1998, U.S. Pat. No. 5,728,375.
Kisilevsky R, Szarek W, Weaver D. “A Method of Treating Amyloidosis”. U.S. Ser. No. 08/463,548, issued Oct. 26, 1999, U.S. Pat. No. 5,972,328.Kisilevsky R, Szarek W, Weaver D. “Method for Treating Amyloidosis”, Japanese patent No. 3,666,818, issued Apr. 15, 2005.
Kisilevsky R, Szarek W, Weaver D. “Method for Treating Amyloidosis”, European Patent No. 0814842, issued Oct. 12, 2005.Kisilevsky R, Szarek W, Weaver D. “Method for Treating Amyloidosis”, (Japanese patent No. 363236; U.S. Pat. No. 5,728,375; U.S. Pat. No. 5,972,328; Japanese patent No. 3,666,818; European Patent No. 0814842; European Patent No. 1060750, issued Dec. 7, 2005.2005)
Kurt M A, Davies D C, Kidd M, Duff K and Howlett. Hyperphosphorylated tau and paired helical filament-like structures in the brains of mice carrying mutant amyloid precursor protein and mutant presenilin-1 transgenes. Neurobiol Dis. 2003. 14: 89-97.
Le Corre, S., Klafki, H. W., Plesnila, N., Hubinger, G., Obermeier, A., Sahagun, H., Monse, B., Seneci, P., Lewis, J., Eriksen, J., Zehr, C., Yue, M., McGowan, E., Dickson, D. W., Hutton, M., Roder, H. M. An inhibitor of tau hyperphosphorylation prevents severe motor impairments in tau transgenic mice. Proc. Natl. Acad. Sci. USA 2006 103(25): 9673-9678.
Lee H G, Perry G, Moreira P I, Garrett M R, Liu Q, Zhu X, Takeda A, Nunomura A, Smith M A. Tau phosphorylation in Alzheimer's disease: pathogen or protector? Trends Mol Med. 2005 April; 11(4):164-9.
Leroy K, Bretteville A, Schindowski K, Gilissen E, Authelet M, De Decker R, Yilmaz Z, Buee L, Brion J P. Early axonopathy preceding neurofibrillary tangles in mutant tau transgenic mice. Am J Pathol. 2007 September; 171(3):976-92.
Lesné S, Koh M T, Kotilinek L, Kayed R, Glabe C G, Yang A, Gallagher M, Ashe K H. A specific amyloid-β protein assembly in the brain impairs memory. Nature 2006. 440: 352-7.
Lewis, J., McGowan, E., Rockwood, J., Melrose, H., Nacaraju, P., Van Slegtenhorst, M., Gwinn-Hardy, K., Murphy, M. P., Baker, M., Yu, X., Duff, K., Hardy, J., Corral, A., Lin, W-L., Yen, S-H., Dickson, D. W., Davies, P., Hutton, M. Neurofibrillary tangles, amyotrophy and progressive motor disturbance in mice expressing mutant (P301L) tau protein. Nature Genetics 2000 25: 402-406.
Liu, L., T. Tapiola, S. K. Herukka, M. Heikkila, and H. Tanila, Abeta levels in serum, CSF and brain, and cognitive deficits in APP+PS1 transgenic mice. Neuroreport, 2003. 14(1): p. 163-6.
Liu, S., I. Ninan, I. Antonova, F. Battaglia, F. Trinchese, A. Narasanna, N. Kolodilov, W. Dauer, R.D. Hawkins, and O. Arancio, alpha-Synuclein produces a long-lasting increase in neurotransmitter release. Embo J, 2004. 23(22): p. 4506-16.
Maeda S, Sahara N, Saito Y, Murayama S, Ikai A, Takashima A. Increased levels of granular tau oligomers: an early sign of brain aging and Alzheimer's disease. Neurosci Res. 2006 March; 54(3):197-201.
Maeda S, Sahara N, Saito Y, Murayama M, Yoshiike Y, Kim H, Miyasaka T, Murayama S, Ikai A, Takashima A. Granular tau oligomers as intermediates of tau filaments. Biochemistry. 2007 Mar. 27;46(12):3856-61.
Makrides V., Shen T. E., Bhatia R., Smith B. L., Thimm J., Lal R., Feinstein S. C. Microtubule dependant oligomerization of tau: Implications of physiological tau-function and taupathies: J. Biol. chem. 2003. 278: 33298-33304
Masliah, E., Mechanisms of synaptic dysfunction in Alzheimer's disease. Histol Histopathol, 1995. 10(2): p. 509-19.
Mayeux R and Sano M. Treatment of Alzheimer's disease. New Engl J Med 1999. 341: 1670-1679.
McLauin J, Kierstead M E, Brown M E, Hawkes C A, Lambermon M H, Phinney A L, Darabie A A, Cousins J E, French J E, Lan M F, Chen F, Wong S S, Mount H T, Fraser P E, Westaway D, St. George-Hyslop P. Cyclohexanehexol inhibitors of Abeta aggregation prevent and reverse Alzheimer phenotype in a mouse model. Nat Med 2006. 12: 801-8.
Mirra S S, Heyman A, McKeel D, Sumi S M, Crain B J, Brownlee L M, Vogel F S, Hughes J P, Van Belle G, Berg L and participating CERAD neuropathologists. The Consortium to Establish a Registry for Alzheimer's Disease (CERAD). Part II. Standardization of the neuropathologic assessment of Alzheimer's disease. Neurology 1991. 41: 479-486.
Moe, J G, Rojas P M, Davidowitz E J, Grossman A. Screening of small molecules that inhibit formation of tau protein oligomers. Society for Neuroscience Meeting 2006.
Nunomura A., Castellani R. J., Lee H., Moreira P. I., Zhu X., Perry G., Smith M. A., Neuropathology in Alzheimer's Disease: Awaking from a Hundred-Year-Old Dream. Sci. Aging Knowl. Environ. 2006 (8), pe10 (2006).
Nussbaum R L, Ellis C E. Alzheimer's disease and Parkinson's disease. New Engl J Med 2003. 348: 1356-1364.
Philips R G and LeDoux J E. Differential contribution of amygdala and hippocampus to cued and contextual fear conditioning. Behay. Neurosci. 106(1992): 274-285.
Puolivali, J., J. Wang, T. Heikkinen, M. Heikkila, T. Tapiola, T. van Groen, and H. Tanila, Hippocampal A beta 42 levels correlate with spatial memory deficit in APP and PS 1 double transgenic mice. Neurobiol Dis, 2002. 9(3): p. 339-47.
Puzzo D, Privitera L, Leznik E, Fà M, Staniszewski A, Palmeri A, Arancio O. Picomolar amyloid-beta positively modulates synaptic plasticity and memory in hippocampus. J Neurosci. 2008, 28(53):14537-45.
Santacruz K, Lewis J, Spires T, Paulson J, Kotilinek L, Ingelsson M, Guimaraes A, DeTure M, Ramsden M, McGowan E, Forster C, Yue M, Orne J, Janus C, Mariash A, Kuskowski M, Hyman B, Hutton M, Ashe K H. Tau suppression in a neurodegenerative mouse model improves memory function. Science. 2005 Jul. 15; 309(5733):476-81
Sant'Angelo, A., F. Trinchese, and O. Arancio, Usefulness of behavioral and electrophysiological studies in transgenic models of Alzheimer's disease. Neurochem Res, 2003. 28(7): p. 1009-15.
Savvateeva-Popova E, Popov A, Grossman A, Nikitina E, Medvedeva A, Peresleni A, Korochkin L, Moe J G. Davidowitz E, Pyatkov K, Myasnyankina E, Zatsepina O, Schostak N, Zelentsova E and Evgen'ev M. Pathogenic chaperone-like RNA induces congophilic aggregates and facilitates neurodegeneration in Drosophila. Cell Stress & Chaperones 2007, In Press.
Selkoe D J. Defining molecular targets to prevent Alzheimer disease. Arch Neurol. 2005. 62: 192-195.
Spires T L, Orne J D, SantaCruz K, Pitstick R, Carlson G A, Ashe K H, Hyman B T., Region-specific dissociation of neuronal loss and neurofibrillary pathology in a mouse model of tauopathy. Am J Pathol. 2006 May; 168(5):1598-607.
Swainson R, Hodges J R, Galton C J, Semple J, Michael A, Dunn B D, Iddon J L, Robbins T W, Sahakian B J. Early detection and differential diagnosis of Alzheimer's disease and depression with neuropsychological tasks. Dement Geriatr Cogn Disord. (2001) 12(4):265-80.
Takahashi, R. H., C. G. Almeida, P. F. Kearney, F. Yu, M. T. Lin, T. A. Milner, and G. K. Gouras, Oligomerization of Alzheimer's beta-amyloid within processes and synapses of cultured neurons and brain. The Journal of Neuroscience, 2004. 24(14): p. 3592-3599.
Townsend M, Shankar G M, Mehta T, Walsh D M, Selkoe D J. Effects of secreted oligomers of amyloid beta-protein on hippocampal synaptic plasticity: a potent role for trimers. J Physiol, 2006. 572(Pt 2): 477-92.
Trinchese, F., S. Liu, F. Battaglia, S. Walter, P. M. Mathews, and O. Arancio, Progressive age-related development of Alzheimer-like pathology in APP/PS1 mice. Ann Neurol, 2004. 55(6): p. 801-14.
Trinchese, F., S. Liu, I. Ninan, D. Puzzo, J. P. Jacob, and O. Arancio, Cell cultures from animal models of Alzheimer's disease as a tool for faster screening and testing of drug efficacy. J Mol Neurosci, 2004. 24(1): p. 15-21.
Vasan S., Mong P. M., Grossman A. Interaction of Prion Protein with small highly structure RNAs: Detection and Characterization of PrP-Oligomers. Neurochemical Research. 2006. 31(5):629-37.
Vasan S., Mong P. M., Grossman A. Interaction of Prion Protein with small highly structure RNAs: Detection and Characterization of PrP-Oligomers. Neurochemical Research. 2006. 31(5):629-37.
Vechterova L., Kontsekova E., Zilka N., Ferencik M., Ravid R and Novak M. DC11:a novel monoclonal antibody revealing Alzheimer's disease-sepcific tau epitope. Genetics of Nervous System Diseases. 2003.14, 87-91.
von Bergen M, Friedhoff P, Biernat J, Heberle J, Mandelkow E M, Mandelkow E. Assembly of tau protein into Alzheimer paired helical filaments depends on a local sequence motif ((306)VQIVYK(311)) forming beta structure. Proc Natl Acad Sci U S A. 2000 May 9; 97(10):5129-34.
Walsh D M, Selkoe D J. Oligomers on the brain: the emerging role of soluble protein aggregates in neurodegeneration. Protein Pept Lett. 2004. 11: 213-228.
Wittman C. W., Wszolek M. F., Shulman J. M., Salvaterra P. M. Lewis J., Hutton M., and Feany M. B. Tauopathy in Drosophila:Neurodegeneration without Neurofibrillary Tangles. Science. 2001. 293, 711-714.
Yoshiyama Y, Higuchi M, Zhang B, Huang S M, Iwata N, Saido T C, Maeda J, Suhara T, Trojanowski J Q, Lee V M. Synapse loss and microglial activation precede tangles in a P301S tauopathy mouse model. Neuron. 2007 53(3):337-51.
Zhang Y. J., Xu Y. F., Chen R. Q., Wang X. C., and Wang J-C. Nitration and oligomerization of tau induced by peroxynitrite inhibit it microtubule binding activity. FEBS Lett. 2005. 579:2421-2427.

What is claimed is:

1. A method for determining whether a test compound can ameliorate tau protein induced reduction of long term potentiation in a neural structure, the method comprising:
(a) contacting a neural structure with a solution containing tau proteins and a test compound,
(b) stimulating long term potentiation between neurons in the neural structure,
(c) measuring long term potentiation between neurons in the neural structure, and
(d) comparing the long term potentiation measured in (c) with long term potentiation measured in neurons in a neural structure contacted with tau proteins in the absence of a compound, wherein an increase in long term potentiation measured in (c) relative to long term potentiation measured in the absence of the compound indicates that the test compound ameliorates tau protein induced reduction of long term potentiation in a neural structure.

2. A method for determining whether a test compound can re-establish or rescue synaptic function in a neural structure following damage by tau proteins, the method comprising:
(a) contacting a neural structure with a solution containing tau proteins and a test compound,
(b) stimulating long term potentiation between neurons in the neural structure,
(c) measuring long term potentiation between neurons in the neural structure, and
(d) comparing the long term potentiation measured in (c) with long term potentiation measured in neurons in a neural structure contacted with tau proteins in the absence of a compound, wherein an increase in long term potentiation measured in (c) relative to long term potentiation measured in the absence of the compound indicates that the test compound re-establishes or rescues synaptic function in a neural structure following damage by tau proteins.

3. A method for determining whether a test compound can increase synaptic function in a neural structure contacted with tau proteins, the method comprising:
(a) contacting a neural structure with a solution containing tau proteins and a test compound,
(b) stimulating long term potentiation between neurons in the neural structure,
(c) measuring long term potentiation between neurons in the neural structure, and
(d) comparing the long term potentiation measured in (c) with long term potentiation measured in neurons in a neural structure contacted with tau proteins in the absence of a compound, wherein an increase in long term potentiation measured in (c) relative to long term potentiation measured in the absence of the compound indicates that the test compound increases synaptic function in a neural structure contacted with tau protein.

4. A method for determining whether a test compound is capable of treating Alzheimer's disease in a subject, the method comprising:
(a) contacting a neural structure with a solution containing tau proteins and a test compound,
(b) stimulating long term potentiation between neurons in the neural structure,
(c) measuring long term potentiation between neurons in the neural structure, and
(d) comparing the long term potentiation measured in (c) with long term potentiation measured in neurons in a neural structure contacted with tau proteins in the absence of a compound, wherein an increase in long term potentiation measured in (c) relative to long term potentiation measured in the absence of the compound indicates that the test compound is capable of treating Alzheimer's disease in a subject.

5. A method for determining whether a test compound can treat a tauopathy disorder in a subject, the method comprising:
(a) contacting a neural structure with a solution containing tau proteins and a test compound,
(b) stimulating long term potentiation between neurons in the a neural structure,
(c) measuring long term potentiation between neurons in the a neural structure, and
(d) comparing the long term potentiation measured in (c) with long term potentiation measured in neurons in a neural structure contacted with tau proteins in the absence of a compound, wherein an increase in long term potentiation measured in (c) relative to long term potentiation measured in the absence of the compound indicates that the test compound is capable of treating a tauopathy disorder in a subject.

6. The method of any of claims 1-5, wherein the neural structure is a brain slice.

7. The method of any of claims 1-5, wherein the neural structure is a hippocampal slice.

8. The method of any of claims 1-5, wherein the neural structure is contacted with the tau protein and test compound serially.

9. The method of any of claims 1-5, wherein the neural structure is contacted with the tau protein and the test compound in parallel.

10. The method of any of claims 1-5, wherein the neural structure is contacted with the tau protein and the test compound for about 30 minutes.

11. The method of any of claims 1-5, wherein the contacting step comprises perfusion of the neural structure.

12. The method of any of claims 1-5, wherein the method comprises an additional step before step (a) of acquiring a baseline long term potentiation reading.

13. The method of any of claims 1-5, wherein the method comprises an additional step of contacting the neural structure with a kinase inhibitor.

14. The method of any of claims 1-5, wherein the method comprises an additional step of contacting the neural structure with a phosphatase inhibitor.

15. A method for determining whether a test compound can treat a learning behavior defect in a subject, the method comprising:
(a) administering a solution containing tau proteins to a first non-human mammal,
(b) administering a test compound to the first non-human mammal,
(c) measuring contextual learning behavior in the first non-human mammal, and
(d) comparing the contextual learning behavior measured in (c) with contextual learning behavior measured in a second non-human mammal, wherein an increase in contextual learning behavior measured in (c) relative to the contextual learning behavior measured second non-human mammal indicates that the test compound is capable of a learning behavior defect in the subject.

16. The method of claim 15, wherein the second non-human mammal is administered with the solution containing tau proteins but not administered the test compound.

17. The method of claim 15, wherein the tau proteins are administered into the hippocampus of the non-human mammal.

18. The method of claim 17, wherein the administration is through a cannula implanted into the hippocampus of the non-human mammal.

19. The method of claim 15, wherein the contextual learning is freezing behavior or fear conditioning.

20. The method of claim 15, wherein the learning behavior defect is Alzheimer's disease.

21. The method of any of claim 1-5 or 15, wherein the concentration of the tau protein in the solution containing tau proteins is about 1 pM to about 5 μM.

22. The method of any of claim 1-5 or 15, wherein the solution containing tau proteins further contains one or more proteins associated with neurodegenerative diseases.

23. The method of any of claim 1-5 or 15, wherein the tau proteins are monomers, soluble oligomers, insoluble aggregates or any combination thereof

24. The method of any of claim 1-5 or 15, wherein the tau proteins are oligomers.

25. The method of any of claim 1-5 or 15, wherein the tau proteins are reactive monomers or reactive oligomers.

26. The method of any of claim 1-5 or 15, wherein the tau proteins are aggregated.

27. The method of any of claim 1-5 or 15, wherein the tau proteins comprise 3R or 4R isoforms or any fragments thereof

28. The method of any of claim 1-5 or 15, wherein the tau proteins comprise tau352, tau 383, tau381, tau410, tau412 and tau 441 or any fragments thereof

29. The method of any of claim 1-5 or 15, wherein the tau proteins are hyperphosphorylated.

30. The method of any of claim 1-5 or 15, wherein the tau proteins comprise a P301L mutation.

31. The method of any of claim 1-5 or 15, wherein the tau proteins are selected from the group consisting of tau 4R/2N, tau 4R/1N, or C-terminal tau 4R.

32. The method of any of claim 1-5 or 15, wherein the tau proteins have an intermolecular disulfide linkage.

33. The method of any of claim 1-5 or 15, wherein the tau proteins have an intramolecular disulfide linkage.

34. The method of any of claim 1-5 or 15, wherein the tau proteins are human Alzheimer's disease tau proteins.

35. The method of any of claim 1-5 or 15, wherein the tau proteins are phosphorylated at tyrosine 217, tyrosine 231, or any combination thereof.

36. The method of any of claim 1-5 or 15, wherein the tau proteins comprise a mutation that increases microtubule binding by the tau protein.

37. The method of any of claim 1-5 or 15, wherein the tau proteins comprise a mutation that decreases microtubule binding by the tau protein.

38. The method of any of claim 1-5 or 15, wherein the test compound is a small molecule, a molecule from a molecular compound library, a protein, a peptide, a nucleic acid, a synthetic molecule, a carbohydrate, a peptidomimetic, a lipid, an antibody or the like.

39. The method of any of claim 1-5 or 15, wherein more than one test compound is contacted to the neural structure.
